(19) United States
(12) Patent Application Publication
(10) Pub. No.: US 2015/0331001 A1
(43) Pub. Date: Nov. 19, 2015

(54) METHODS AND APPARATUS FOR THE ANALYSIS OF FATTY ACIDS

(71) Applicant: WATERS TECHNOLOGIES CORPORATION, Milford, MA (US)

(72) Inventors: Michael D. Jones, Narragansett, RI (US); Giorgis Mezengie Isaac, Marlborough, MA (US); Isabelle Francois, Sint-Lievens-Houtem (BE); Warren B. Potts, Fitchburg, MA (US); James I. Langridge, Sale (GB)

(21) Appl. No.: 14/762,997

(22) PCT Filed: Jan. 24, 2014
(86) PCT No.: PCT/US14/12894

§ 371 Date: Jul. 23, 2015

Related U.S. Application Data
(60) Provisional application No. 61/756,865, filed on Jan. 25, 2013.

(51) Int. Cl. G01N 33/92; B01D 15/40; B01D 15/42

(52) U.S. Cl. CPC G01N 33/92; B01D 15/424; B01D 15/40; G01N 2560/00; G01N 2405/08; G01N 2405/04; G01N 2405/02

(57) ABSTRACT

Exemplary embodiments of the present disclosure relate to CO₂-based chromatography for the efficient and precise separation of fatty acids. The present disclosure is based, in part, on the discovery that a CO₂-based chromatography system with features, such as, e.g., improved pressure stability, improved sample injection, and superior column packing materials, reproducibly resolve fatty acids.

### RELATED APPLICATIONS

This application claims the benefit of U.S. Provisional Application No. 61/756,865, filed Jan. 25, 2013, the entire contents of which are incorporated herein by reference.

### FIELD OF THE TECHNOLOGY

The present disclosure relates to CO₂-based chromatography for use in the rapid qualitative and quantitative analysis of fatty acids.

### BACKGROUND

Lipids play a variety of cellular roles and are the principal form of stored energy in most organisms. Specialized lipids serve as pigments, cofactors, detergents, transporters, hormones, extracellular and intracellular messengers, and anchors for membrane proteins. Fatty acids are key constituent of lipids. Lipids possess their hydrophobicity because of their fatty acid makeup, therefore providing a necessary tool in the formation of membranes. In nature, most fatty acids exist as straight-chain hydrocarbons that attach to a carboxylic acid. When double bonds are present, fatty acids are defined as unsaturated, monounsaturated (if one double bond is present), or polyenoic (if two or more double bonds are generally separated by a single methylene group in the carbon backbone).

Routine determinations of free fatty acids, especially in plasma, often measure the total amount of fatty acids in a sample and not the individual fatty acids that are present. This is particularly true with conventional chromatographic methods. The general assumption that one particular fatty acid is representative of all fatty acids is an important limitation of this approach. Due to the inherent separation power, chromatographic methods are capable of quantitatively analyzing individual fatty acids in both commercial and biological samples.

Typical chromatographic methods for analyzing fatty acids include gas chromatography/mass spectroscopy (GC/MS) and liquid chromatography-tandem mass spectrometry (LC/MS/MS). However, there are shortcomings associated with each of these methods. For example, GC methods require derivatization of the fatty acids to methyl esters (FAME), which is burdensome, time consuming, and leaves doubt as to whether the esters formed are from free fatty acids or intact complex lipids. In LC/MS/MS methods, although no sample derivatization is required, separations typically involve labor intensive and time consuming sample preparation, and utilize toxic organic solvents, which are expensive to purchase and dispose of.

The use of non-toxic CO₂ as an alternative to organic solvents as the mobile phase has resulted in the advent of CO₂-based chromatography. The CO₂ alone, or in combination with a co-solvent or modifier, provides a low viscosity mobile phase that achieves higher diffusion rates and enhanced mass transfer over the solvents used in HPLC. While chromatographic systems utilizing CO₂ as a mobile phase constituent have been considered, prior systems suffer from supercritical fluid chromatography systems suffer from, e.g., long sample run time, susceptibility to system pressure fluctuations causing sample backflow, sample carryover, and lack of robustness, all of which prevent users from obtaining reproducible results.

### SUMMARY

Due to the valuable properties of fatty acids in the biosynthesis of lipids, and for commercial applications, e.g., in the area of polyamide resins, there is a need to develop improved chromatography systems and methods that overcome the above limitations and allow for rapid and robust analysis of fatty acids.

Thus, exemplary embodiments of the present disclosure are directed to rapid and efficient methods for the separation and analysis of fatty acids. The present disclosure is based, in part, on the discovery that a CO₂-based chromatography system (e.g., ACQUITY UPC²®, Waters Corporation, Milford, Mass.) with features, such as, e.g., improved pressure stability, improved sample injection, and superior column packing materials, could reproducibly substantially resolve fatty acids.

The present disclosure is also based, in part, on the discovery that improved pressure stability, achieved from the methods comprising the described CO₂-based chromatography systems, allows for the implementation of smaller average particle sized columns of various lengths and diameters. As described herein, at least one contributing factor for the superior separations achieved with one or more fatty acids is the ability to use, and the inclusion of, smaller average particle sized columns. For example, columns with average particle sizes of 2 microns or less can be used with the described CO₂-based chromatography systems without limiting the resolution of one or more fatty acids.

Without being bound by theory, particle stationary phases having average particle sizes of 2 microns or less provide increases in efficiency that are typically measured by Height Equivalent Theoretical Plates (HETP) or for gradient chromatography, calculation of the chromatographic peak capacity which is dictated by the width of the eluting bands per unit time. In one aspect, to realize the chromatographic benefits provided by average particle sizes of 2 microns or less, the resolution equation (Purnell's Equation) suggests that chromatographic instrumentation be optimized to mitigate detrimental effects of extra column volume. Previous instrumentations, however, have not been designed to mitigate these detrimental effects and, therefore, have been not realized or have been unsuccessful in obtaining enhanced separation methods and processes.

Yet, as described herein, and in addition to other factors, smaller particle sizes used in connection with the present systems increase chromatographic efficiency, which in turn provides improvements in resolution between eluting bands of one or more fatty acids. In addition, smaller particles also provide increases in linear velocity which has been found to decrease the speed of analysis. In combination, these effects lead to the present disclosure of chromatographic methods and processes with superior sensitivity.

In addition to other advantages, the CO₂-based chromatography methods described herein minimize consumption of mobile phase solvents (e.g., methanol) thereby generating less waste for disposal and reducing the cost of analysis per sample. Also, because relatively short chromatographic run times (less than 5 minutes) are typically achieved with effective separation, the unique speed and resolution provided by the CO₂-based chromatography methods described herein serve as a key element in developing high-throughput routine screening assays.

The apparatus and methods described herein further comprise, at least in part, an efficient and precise method for the analysis of fatty acids using CO₂-based chromatography. In some embodiments, at least a portion of CO₂ is in supercritical state (or near supercritical state).

### BRIEF DESCRIPTION OF THE DRAWINGS

The foregoing and other features and advantages provided by the present disclosure will be more fully understood from the following description of exemplary embodiments when read together with the accompanying drawings, in which:

FIG. 1 is an exemplary graph of the physical state of a substance in relation to a temperature and pressure associated with the substance;

FIG. 2 is a schematic view of a CO₂-based chromatography system as described herein;

FIG. 3 is a block diagram of an exemplary arrangement of an embodiment of the system of FIG. 2;

FIG. 4 is a block diagram of another exemplary arrangement of an embodiment of the system of FIG. 2;

FIG. 5 is a flow diagram of a mobile phase through a system manager portion of the an exemplary embodiment of the CO₂-based chromatography system;

FIG. 6 is a cross-sectional view of a valve assembly for an exemplary dynamic pressure regulator in an exemplary embodiment of the CO₂-based chromatography system;

FIG. 7 is an exploded perspective view of an exemplary embodiment of a calibration collar according to the present disclosure;

FIG. 8 is a perspective view of another exemplary embodiment of the calibration collar according to the present disclosure; in this view the calibration collar is shown without fasteners;

FIG. 9 is a cross-sectional view of another exemplary dynamic pressure regulator; in this view the dynamic pressure regulator includes the calibration collar of FIG. 8;

FIGS. 10(a)-(b) are side and detailed views of an exemplary embodiment of an interior portion of a shaft of an actuator included in the pressure regulator shown in FIG. 6;

FIG. 11 represents an exemplary embodiment of a portion of FIG. 6, which is a cross-sectional view of a portion of the dynamic pressure regulator;

FIG. 12 is an exemplary embodiment of a vent valve according to the present disclosure;

FIGS. 13 (a) and (b) are exemplary embodiments of a seat according to the present disclosure;

FIG. 14 illustrates an exemplary embodiment of a needle with stem grooves and exemplary seat plastic deformation according to the present disclosure;

FIG. 15 is an exemplary embodiment of a seat retainer assembly illustrating a pressure assist according to the present disclosure;

FIG. 16 is an exemplary embodiment of a vent valve in an open position according to the present disclosure;

FIG. 17 represents a pressure regulator heating system according to an embodiment of the present disclosure, where a) illustrates a heating element and a static pressure regulator, and b) illustrates the system with the static pressure regulator removed;

FIG. 18 represents a static pressure regulator according to one embodiment of the present disclosure;

FIG. 19 is an exemplary CO₂-based chromatography system analysis of nine fatty acids (ranging from C₈ to C₂₄);

FIG. 20 illustrates the impact of backpressure on the sensitivity of the analysis for a panel of fatty acids;

FIG. 21 illustrates the impact of varying solvent gradient conditions on a panel of fatty acids;

FIG. 22 illustrates the impact of higher percentages of co-solvents on gradient separations on a panel of fatty acids;

FIG. 23 illustrates a representative chromatogram from the analysis of algaenan 1;

FIG. 24 illustrates a Principal component analysis (PCA) of algae and algaenan oil extracts;

FIGS. 25(a)-25(d) illustrate comparative plots between algae 1 vs. algae 2 where (a) represents an orthogonal projections latent structure discriminant analysis (OPLS-DA) between algae 1 and algae 2 group difference; (b) represents an S-plot indicating the features that contribute to the group difference between algae 1 and algae 2; (c) provides a representative trend plot showing the major up-regulated 16:1, 18:1, and 24:0 free fatty acids in algae 1; and (d) provides a representative trend plot showing the major up-regulated 8:0, 13:0, and 24:1 free fatty acids in algae 2;

FIG. 26a illustrates an ion map, mass spectrum, and chromatogram across all runs for a selected free fatty acid 20:0;

FIG. 26b illustrates a normalized abundance of free fatty acid 29:0;

FIG. 27 illustrates an analysis of a mouse heart extract;

FIG. 28 illustrates chromatograms from an extracted blood sample;

FIG. 29 illustrates the Ultraviolet (UV) chromatograms of Triacylglycerols in peanut, sunflower seed, and soybean oil; and

FIG. 30 illustrates baseline separation of glycerol, soybean oil acylglycerols, and model biodiesel components.

### DETAILED DESCRIPTION

In an embodiment, the present disclosure provides a method of separating one or more fatty acids, comprising: placing a sample (e.g., a biological sample or commercial sample) in a CO₂-based chromatography system comprising a chromatography column packed with particles having a mean particle size of 0.5 to 3 microns (e.g., of 2 microns or less, about 1.7 microns, or about 1.8 microns); and eluting the sample with organic solvent and a mobile phase fluid comprising CO₂ to substantially resolve the one or more fatty acids.

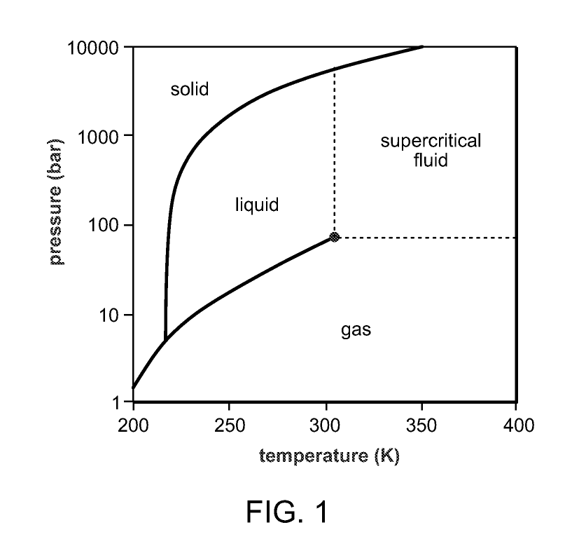

With reference to FIG. 1, the terms “near supercritical state” or “supercritical fluid” mean a state or phase of a substance (such as CO₂ or CO₂ with a modifier such as methanol) that is close to, but does not necessarily fall on or within the supercritical region represented by the dotted lines in FIG. 1. Thus, these terms are intended to encompass the state of a substance which comprises one or more of the advantageous properties of being in the supercritical state or in a substantially supercritical fluid form (i.e., on, within, or near the dotted lines in FIG. 1), while not necessarily objectively falling into a pressure and temperature range that correlates to being on or within the supercritical fluid region set forth by the dotted lines in FIG. 1. It would also be understood that instrumentation settings for the CO₂-based chromatography systems described herein impact the nature of being at or near supercritical state, or producing or maintaining supercritical (or near supercritical) fluid.

As modifiers such as methanol are typically added, particularly when using gradients, the mobile phase may not be maintained within supercritical state. However, the high pressures and pressure control associated with the CO₂-based chromatography system described herein (e.g., ACQUITY UPC²®, Waters Corporation, Milford, Mass.), provide the advantageous features associated with supercritical fluids, such that these near supercritical fluids provide substantially the same advantageous properties.

For example, for CO₂, and without making certain modifications (e.g., tubing modifications) of the CO₂-based chromatography systems described herein, a pressure value above approximately 1070.4 psi (or 73.8 bar) is needed to maintain the supercritical state, or effects thereof, during the analysis of one or more fatty acids. It would further be understood that in the event of no pressure cut off limits (e.g., below about 1000 or above about 9000 psi), the CO₂-based chromatography systems described herein, particularly the pump component, would not effectively compress the CO₂, such that air would be captured inside the pump chamber and ultimately lead to no CO₂ delivery as the CO₂ would presumably be in gas form as opposed to at or near supercritical state. Thus, as further discussed and shown in detail below (see e.g., the exemplary embodiments discussed below of certain individual components comprised in the CO₂-based chromatography systems described herein), pressure, and in particular the maintenance, steady control, and decreased fluctuations thereof, is one of the factors for maintaining, or otherwise obtaining the benefits of being at or near the supercritical state of CO₂ during the analysis of one or more fatty acids. The present CO₂-based chromatography system (e.g., ACQUITY UPC²®, Waters Corporation, Milford, Mass.) comprises, as one of its many superior features, improved pressure stability.

As used herein, the term “biological sample” refers to any solution or extract containing a molecule or mixture of molecules that comprise at least one biomolecule that is subjected to analysis and which originated from a biological source. It would be understood that the biological sample may or may not contain one or more fatty acids as it would be apparent that the methods described herein prove useful in determining the presence or absence of one or more fatty acids contained in a given sample. Biological samples are intended to include crude or purified, e.g., isolated or commercially obtained, samples. Particular examples include, but are not limited to, solutes, inclusion bodies, biological matrices, embedded tissue samples, cells (e.g., one or more types of cells), cell culture supernatants, tissues, fluids, and extracts from in vitro and in vivo samples (e.g., plants, seeds, animals, humans, etc.). The sample may further include macromolecules, e.g., substances, such as biopolymers, e.g., proteins, e.g., proteolytic proteins or lipophilic proteins, such as receptors and other membrane-bound proteins, and peptides.

As used herein, the term “commercial sample” refers to samples used for commercial purposes, such as, in the production of certain goods that are not intended as therapeutics for the treatment of diseases or disorders. It would be understood that the commercial sample may or may not contain one or more fatty acids as it would be apparent that the methods described herein prove useful in determining the presence or absence of one or more fatty acids contained in a given sample. Commercial samples include, e.g., combustible organic material, fossil fuels, drop-in fuels (such as algae-based fuels), polymers for inorganic product compositions, and food and healthcare products such as, e.g., oils, vitamins, cosmetics (e.g., perfumes, shampoos, hair products, creams, ointments, etc.), fruits, meats, vegetables, petroleum jellies, and fat and water based gels.

It should also be noted that a biological or commercial sample as described herein can be treated to remove components that could interfere with the detection of the presence or absence of one or more fatty acids. A variety of techniques known to those having skill in the art can be used based on the sample type. For example, solid and/or tissue samples can be ground and extracted to free the analytes of interest from interfering components. In such cases, a sample can be centrifuged, filtered, and/or subjected to chromatographic techniques to remove interfering components (e.g., cells or tissue fragments). In yet other cases, reagents known to precipitate or bind the interfering components can be added. For example, whole blood samples can be treated using conventional clotting techniques to remove red and white blood cells and platelets. A sample can be also be de-proteinized. For example, a plasma sample can have serum proteins precipitated using conventional reagents such as acetonitrile, KOH, NaOH, or others known to those having ordinary skill in the art, optionally followed by centrifugation of the sample. Moreover, a sample can be subject to extraction or derivatization processes to remove or deter unwanted biproducts or components that otherwise affect analysis.

In certain embodiments, an internal standard can be added to a sample prior to sample preparation. Internal standards can be useful to monitor extraction/purification efficiency. An internal standard can be any compound that would be expected to behave under the sample preparation conditions in a manner similar to that of one or more of the analytes of interest. For example, a stable-isotope-labeled version of one or more fatty acids of interest can be used, such as a deuterated version of a fatty acid of interest. While not being bound by any theory, the physicochemical behavior of such stable-isotope-labeled compounds with respect to sample preparation and signal generation would be expected to be identical to that of the unlabeled analyte, such as a fatty acid, but which is differentiable by one or more means, e.g., by mass on a mass spectrometer.

To improve run time and minimize hands-on sample preparation, on-line extraction and/or analytical chromatography of a sample can be used. For example, in certain methods, a sample comprising one or more fatty acids, such as a deproteinized plasma sample, can be extracted using an extraction column, followed by elution onto an analytical chromatography column. The columns can be useful to remove interfering components as well as reagents used in earlier sample preparation steps (e.g., to remove reagents such as acetonitrile). Systems can be coordinated to allow the extraction column to be running while an analytical column is being flushed and/or equilibrated with solvent mobile phase, and vice-versa, thus improving efficiency and run-time. A variety of extraction and analytical columns with appropriate solvent mobile phases and gradients can be chosen by those having ordinary skill in the art.

The methods described herein may further comprise obtaining a mass spectrometer signal of the one or more fatty acids.

In an embodiment, the fatty acids comprise aliphatic tails comprising from about 6 to about 46 carbon atoms, particularly from about 6 to about 26 carbon atoms.

### CO₂-Based Chromatography System

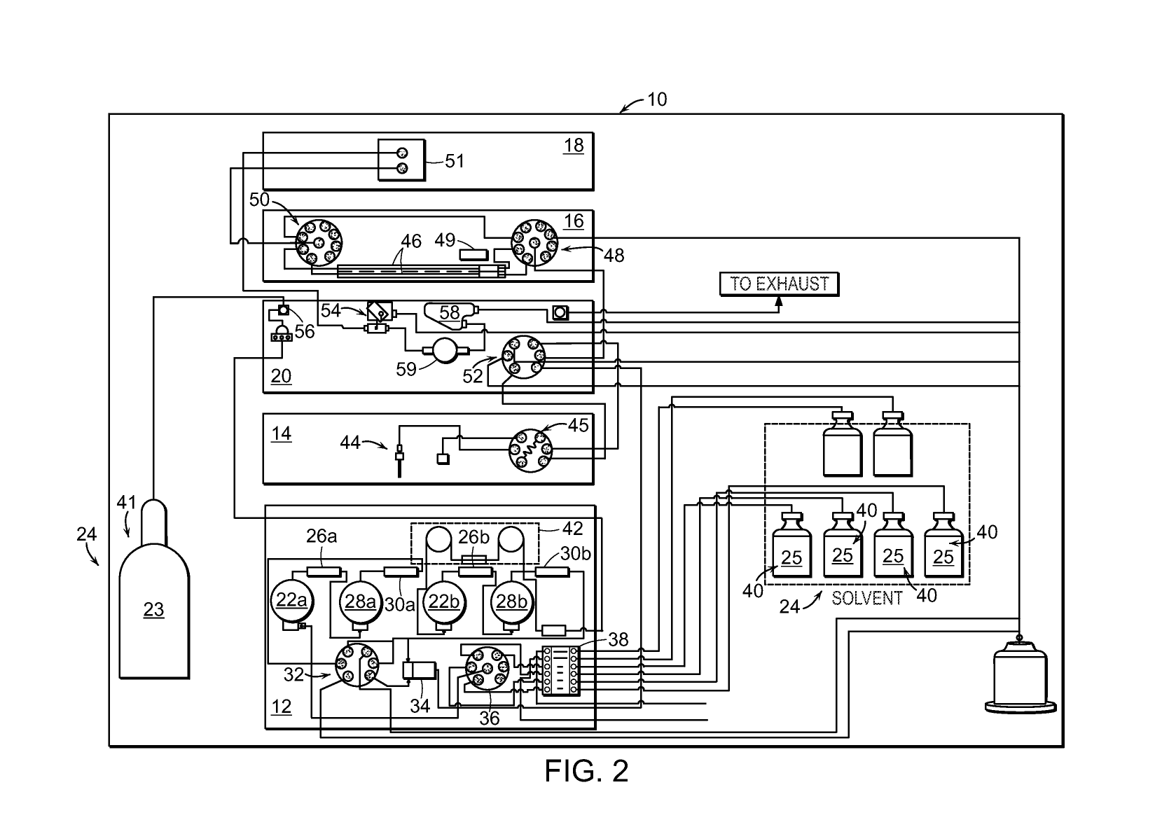

FIG. 2 is a block diagram of an exemplary pressurized flow system, which in the present disclosure is implemented as a CO₂-based chromatography system 10. While the present disclosure is illustrative of a CO₂-based chromatography system, those skilled in the art will recognize that exemplary embodiments of the present disclosure can be implemented as other pressurized flow systems and that one or more system components of the present disclosure can be implemented as components of other pressurized systems. The CO₂-based chromatography system 10 can be configured to detect sample components of a sample using chromatographic separation in which the sample is introduced into a mobile phase that is passed through a stationary phase. The CO₂-based chromatography system 10 can include one or more system components for managing and/or facilitating control of the physical state of the mobile phase, control of the pressure of the CO₂-based chromatography system 10, introduction of the sample to the mobile phase, separation of the sample into components, and/or detection of the sample components, as well as venting of the sample and/or mobile phase from the CO₂-based chromatography system 10.

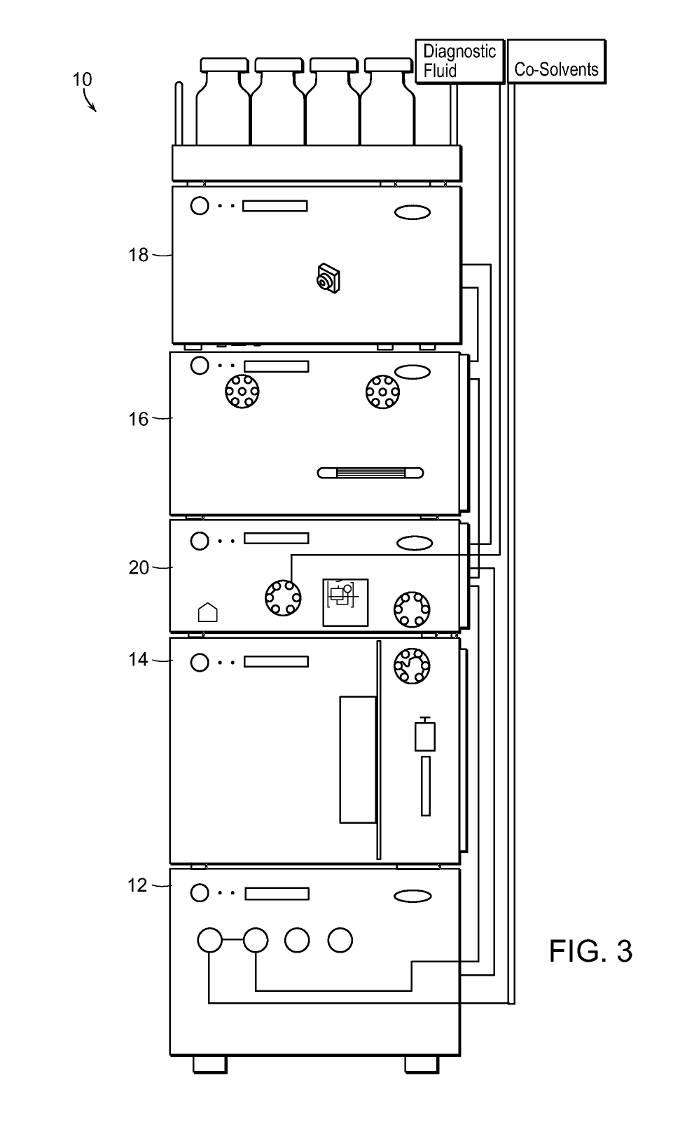

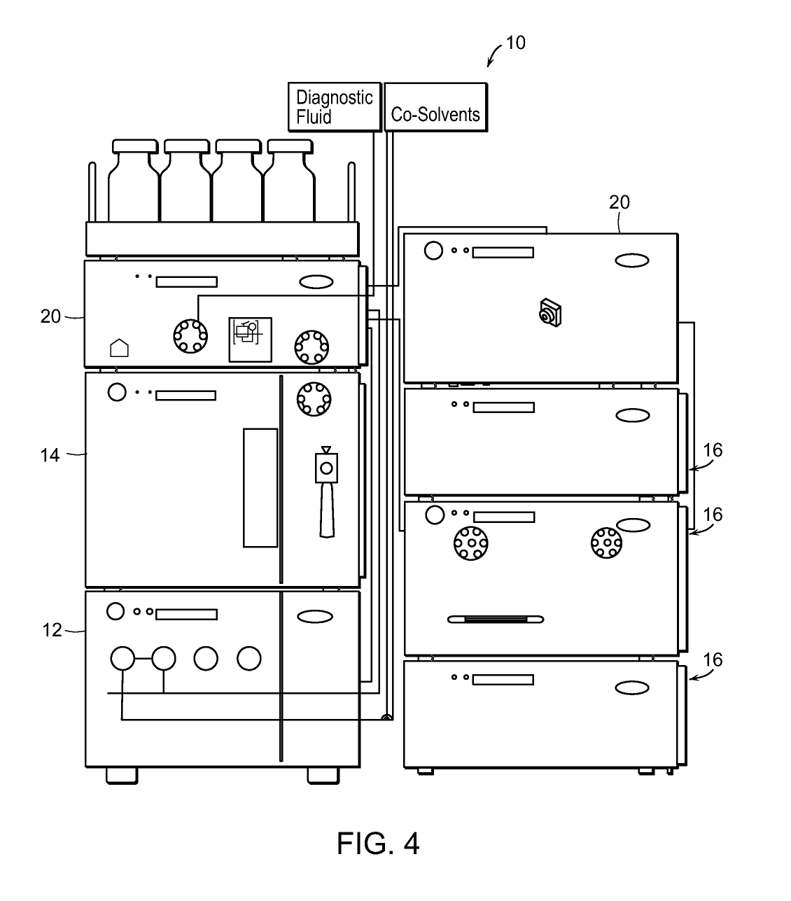

In the present disclosure, the CO₂-based chromatography system 10 can include a solvent delivery system 12, a sample delivery system 14, a sample separation system 16, a detection system 18, and a system/convergence manager 20. In some embodiments, the system components can be arranged in one or more stacks. As another example, the system components of the CO₂-based chromatography system 10 can be arranged in a single vertical stack (FIG. 3). Alternatively, the system components of the CO₂-based chromatography system 10 can be arranged in multiple stacks (FIG. 4). Those skilled in the art will recognize that other arrangements of the components of the CO₂-based chromatography system 10 are possible. Furthermore, while embodiments of the CO₂-based chromatography system 10 have been illustrated as including system components 12, 14, 16, 18, and 20, those skilled in the art will recognize that embodiments of the CO₂-based chromatography system 10 can be implemented as a single integral unit, that one or more components can be combined, and/or that other configurations are possible.

The solvent delivery system 12 can include one or more pumps 22a and 22b configured to pump one or more solvents 24, such as mobile phase media 23 (e.g., carbon dioxide) and/or modifier media 25 (i.e., a co-solvent, such as e.g., methanol, ethanol, 2-methoxyethanol, isopropyl alcohol, or dioxane), through the CO₂-based chromatography system 10 at a predetermined flow rate. For example, the pump 22a can be in pumping communication with the modifier media 25 to pump the modifier media 25 through the CO₂-based chromatography system 10, and the pump 22b can be in pumping communication with the mobile phase media 23 to pump the mobile phase media 23 through the CO₂-based chromatography system 10. An output of the pump 22a can be monitored by a transducer 26a and an output of the pump 22b can be monitored by a transducer 26b. The transducers 26a and 26b can be configured to sense the pressure and/or flow rate associated with the output of the solvent 24 from the pumps 22a and 22b, respectively.

The outputs of the pumps 22a and 22b can be operatively coupled to an input of accumulators 28a and 28b, respectively. The accumulators 28a and 28b are refilled by the outputs of the pumps 22a and 22b, respectively, and can contain an algorithm to reduce undesired fluctuations in the flow rate and/or pressure downstream of the pumps 22a and 22b, which can cause detection noise and/or analysis errors on the CO₂-based chromatography system 10. An output of the accumulator 28a can be monitored by a transducer 30a and an output of the accumulator 28b can be monitored by a transducer 30b. The transducers 30a and 30b can be configured to sense pressure and/or flow rate at an output of the accumulators 28a and 28b, respectively. The outputs of the accumulators 28a and 28b can be operatively coupled to a multiport valve 32, which can be controlled to vent the solvent 24 (e.g., mobile phase media 23 and modifier media 25) being pumped by the pumps 22a and 22b and/or to output the solvent 24 to a mixer 34. The mixer 34 can mix the modifier media 25 and the mobile phase media 23 output from the pumps 22a and 22b, respectively (e.g., after first passing through the accumulators 28a and 28b) and can output a mixture of the mobile phase media 23 and the modifier media 25 to form a solvent stream (i.e., mobile phase) that flows through the CO₂-based chromatography system 10. The output of the mixer 34 can be operatively coupled to the system/convergence manager 20 as discussed in more detail below.

In exemplary embodiments, the solvent delivery system 12 can include a multiport solvent selection valve 36 and/or a degasser 38. The solvent selection valve 36 and/or the degasser 38 can be operatively disposed between an input of the pump 22a and solvent containers 40 such that the solvent selection valve 36 and/or the degasser 38 are positioned upstream of the pump 22a. The solvent selection valve 36 can be controlled to select the modifier media 23 to be used by the CO₂-based chromatography system 10 from one or more solvent containers 40 and the degasser 38 can be configured to remove dissolved gases from the media modifier 23 before the media modifier 23 is pumped through the CO₂-based chromatography system 10.

In exemplary embodiments, the solvent delivery system 12 can include a pre-chiller 42 disposed between an input of the pump 22b and a solvent container 41 such that the pre-chiller is disposed upstream of the input to the pump 22b and downstream of the solvent container 41. The pre-chiller 42 can reduced the temperature of the mobile phase media 23 before it is pumped through the CO₂-based chromatography system 10 via the pump 22b. In the present disclosure, the mobile phase media 23 can be carbon dioxide. The pre-chiller can decrease the temperature of the carbon dioxide so that the carbon dioxide is maintained in a liquid state (i.e., not a gaseous state) as it is pumped through at least a portion of the CO₂-based chromatography system 10. Maintaining the carbon dioxide in a liquid state can facilitate effective metering of the carbon dioxide through the CO₂-based chromatography system 10 at the specified flow rate.

The pumps 22a and 22b can pump the solvent 24 through the CO₂-based chromatography system 10 to system to a specified pressure, which may be controlled, at least in part, by the system/convergence manager 20. In exemplary embodiments, the CO₂-based chromatography system 10 can be pressurized to a pressure between about 700 psi and about 18000 psi or about 1400 psi and about 9000 psi. By pressurizing the CO₂-based chromatography system 10 at these pressure levels (such as those pressure levels described above), the solvent stream (i.e., mobile phase) can be maintained in a liquid state before transitioning to, at or near, supercritical fluid state for a chromatographic separation in a column, which can be accomplished by raising the temperature of the pressurized solvent stream.

The sample delivery system 14 can select samples having one or more fatty acids to be passed through the CO₂-based chromatography system 10 for chromatographic separation and detection. The sample delivery system 14 can include a sample selection and injection member 44 and a multi-port valve 45. The sample selection and injection member 44 can include a needle through which the sample can be injected into the CO₂-based chromatography system 10. The multiport valve 45 can be configured to operatively couple the sample selection and injection member 44 to an input port of the system/convergence manager 20.

The sample separation system 16 can receive the fatty acid sample to be separated and detected from the sample delivery system 14, as well as the pressurized solvent stream from the solvent delivery system 12, and can separate components of the sample passing through the CO₂-based chromatography system 10 to facilitate detection of one or more fatty acids using the detection system 18. The sample separation system 16 can include one or more columns 46 disposed between an inlet valve 48 and an outlet valve 50. The one or more columns 46 can have a generally cylindrical shape that forms a cavity, although one skilled in the art will recognize that other shapes and configurations of the one or more columns is possible. The cavity of the columns 46 can have a volume that can at least partially be filled with retentive media, such as hydrolyzed silica, such as C₈ or C₁₈, or any hydrocarbon to form the stationary phase of the CO₂-based chromatography system 10 and to promote separation of the components of the sample. The inlet valve 48 can be disposed upstream of the one or more columns can be configured to select which of the one or more columns 46, if any, receives the sample. The outlet valve 50 can be disposed downstream of the one or more columns 46 to selectively receive an output from the one or more columns 46 and to pass the output of the selected one or more columns 46 to the detection system 18. The columns 46 can be removably disposed between the valves 48 and 50 to facilitate replacement of the one or more columns 46 to new columns after use. In some embodiments, multiple sample separation systems 16 can be included in the CO₂-based chromatography system 10 to provide an expanded quantity of columns 46 available for use by the CO₂-based chromatography system 10 (FIG. 4). Exemplary columns useful for the separation of one or more fatty acids are discussed in further detail below.

In exemplary embodiments, the sample separation system 16 can include a heater 49 to heat the pressurized solvent stream 24 prior and/or while the pressured solvent stream 24 passes through the one or more columns 46. The heater 49 can heat the pressurized solvent stream to a temperature at which the pressured solvent transitions from a liquid state to at or near supercritical fluid state so that the pressurized solvent stream passes through the one or more columns 46 as a supercritical, or near supercritical fluid.

Referring again to FIG. 2, the detection system 18 can be configured to receive components separated from a sample of one or more fatty acids by the one or more columns 46 and to detect a composition of the components for subsequent analysis. In an exemplary embodiment the detection system 18 can include one or more detectors 51 configured to sense one of more characteristics of the sample components. For example, in one embodiment, the detectors 51 can be implemented as one or more photodiode arrays.

The system/convergence manager 20 can be configured to introduce a sample from the sample delivery system 14 into the pressurized solvent stream flowing from the solvent delivery system 12 and to pass the solvent stream and sample to the sample separation system 16. In the present disclosure, the system/convergence manager 20 can include a multiport auxiliary valve 52 which receives the sample injected by the sample delivery system 14 through a first inlet port and the pressurized solvent stream from the solvent delivery system 12 through a second inlet port. The auxiliary valve 52 can mix the sample and the solvent stream and output the sample and solvent stream via an outlet port of the multiport auxiliary valve 52 to an inlet port of the inlet valve 48 of the sample separation system 16.

The system/convergence manager 20 can also be configured to control the pressure of the CO₂-based chromatography system 10 and to facilitate venting of the solvent from the CO₂-based chromatography system 10, and can include a vent valve 54, a shut off valve 56, a back pressure regulator 58, and a transducer 59. The vent valve 54 can be disposed downstream of the detection system 18 can be configured to decompress the CO₂-based chromatography system 10 by venting the solvent from the CO₂-based chromatography system 10 after the solvent has passed through the CO₂-based chromatography system 10. The shut-off valve 56 can be configured to disconnect the solvent supply from the inlet of the pump 22b of the solvent delivery system to prevent the solvent from being pumped through the CO₂-based chromatography system 10. An exemplary vent valve 54 will be described in more detail below.

The back pressure regulator 58 can control the back pressure of the CO₂-based chromatography system 10 to control the flow of the mobile phase and sample through the column, to maintain the mobile phase in the supercritical fluid state as the mobile phase passes through the one or more columns 46 of the sample separation system 16, and/or to prevent the back pressure from forcing the mobile phase reversing its direction a flow through the one or more columns 46. Embodiments of the back pressure regulator 58 can be configured to regulate the pressure of the CO₂-based chromatography system 10 so that the physical state of the solvent stream (i.e., mobile phase) does not change uncontrollably upstream of and/or within the back pressure regulator 58. The transducer 59 can be a pressure sensor disposed upstream of the back pressure regulator 58 to sense a pressure of the system 10. The transducer 59 can output a feedback signal to a processing device which can process the signal to control an output of an actuator control signal from the processing device.

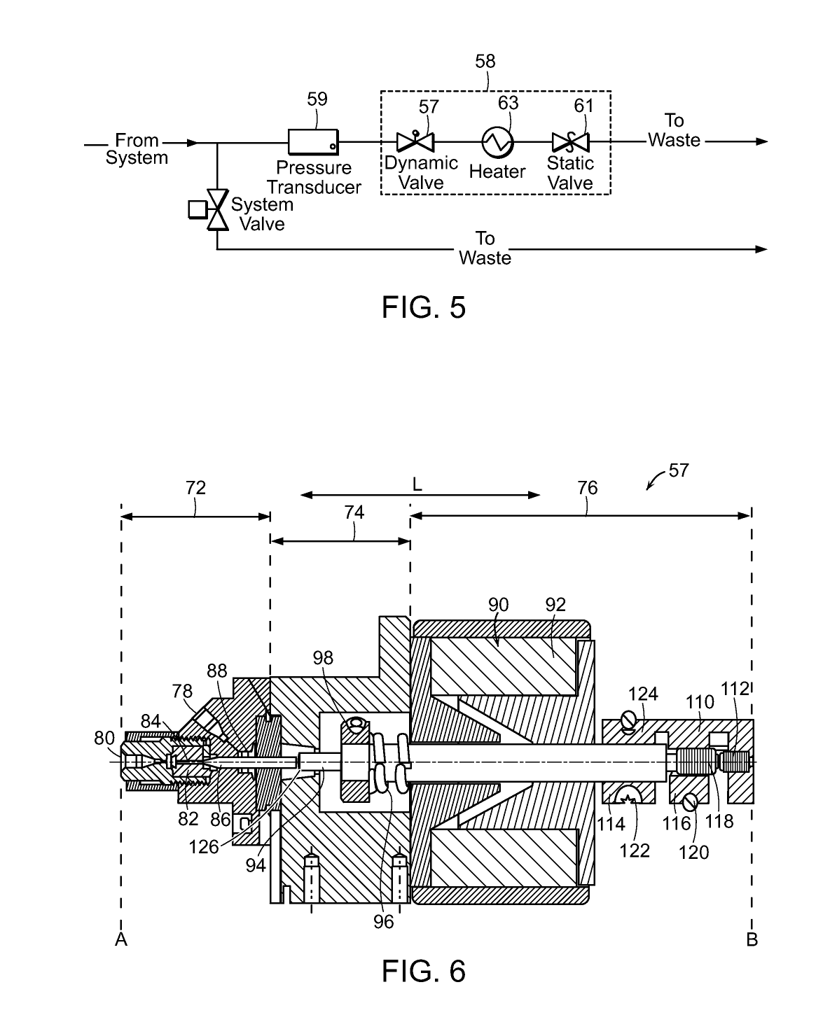

In exemplary embodiments, as shown in FIG. 5, the back pressure regulator 58 can include a dynamic pressure regulator 57, a static pressure regulator 61, and a heater 63. The static pressure regulator 61 can be configured to maintain a predetermined pressure upstream of the back pressure regulator 58. The dynamic pressure regulator 57 can be disposed upstream of the static pressure regulator 61 and can be configured to set the system pressure above the predetermined pressure maintained by the static regulator 61. The heater 63 can be disposed downstream of the dynamic pressure regulator 57 and can be disposed in close proximity to the static pressure regulator 61 to heat the solvent stream as it passes through the static pressure regulator 61 to aid in control of the physical state of the solvent as it passes through the static pressure regulator.

In summary, an exemplary operation of the CO₂-based chromatography system 10 can pump mobile phase media 23 and modifier media 25 at a specified flow rate through the CO₂-based chromatography system 10 as a solvent stream (i.e., mobile phase) and can pressurize the CO₂-based chromatography system 10 to a specified pressure so that the solvent stream maintains a liquid state before entering the sample separation system 16. A sample can be injected into the pressurized solvent stream by the sample delivery system 14, and the sample being carried by the pressurized solvent stream can pass through the sample separation system 16, which can heat the pressurized solvent stream to transition the pressurized solvent stream from a liquid state to a supercritical (or near supercritical) fluid state. The sample and the supercritical fluid (or near supercritical fluid) solvent stream can pass through at least one of the one or more columns 46 in the sample separation system 16 and the column(s) 46 can separate components of the sample from each other. The separated components can pass the separated components to the detection system 18, which can detect one or more characteristics of the sample for subsequent analysis. After the separated sample and solvent pass through the detection system 18, the solvent and the sample can be vented from the CO₂-based chromatography system 10 by the system/convergence manager 20.

In other embodiments, the CO₂-based chromatography system described herein can also be used for preparatory methods and separations. Typical parameters, such as those described above, may be manipulated to achieve effective preparatory separations. For example, the CO₂-based chromatography system described herein confers the benefit of exerting higher flow rates, larger columns, and column packing size, each of which contributes to achieving preparatory separation and function, while maintaining little or no variability in overall peak shape, peak size, and/or retention time(s) when compared to respective analytical methods and separations thereof. Thus, in one embodiment, the present disclosure provides CO₂-based chromatography systems which are amendable to preparatory methods and separations with high efficiency and correlation to analytical runs.

Further detailed reference will now be made to certain components of the CO₂-based chromatography systems of the present disclosure, one or more of which contribute to the advantageous features of the systems and methods described herein, e.g., improved pressure stability, improved sample injection, enhanced sensitivity, improved resolution and retention times, robustness, and consistent reproducibility of sample runs and results obtained therefrom. However, it would be understood that the following description, and components referred to therein, are in no way limiting or exhaustive and are intended to further illustrate the beneficial and superior results obtained by the CO₂-based chromatography systems of the present disclosure.

### Mechanically Self Calibrating Needle Valve

Needles and/or their associated seats in pressurized flow systems can wear out over time and can require replacement or reconfiguration for a different application. Due to tolerances needed for adequate pressure control, the positioning of the needle relative to the seat is generally calibrated after a maintenance event or prior to a start-up condition. As described in U.S. Provisional Application No. 61/607,930 and PCT/US2013/029580, the contents of which are incorporated herein by reference, the CO₂-based chromatography systems described herein automatically sets the position of a needle in a needle valve device used in the CO₂-based chromatography system 10. Mechanical means, such as, for example, springs and locking mechanisms are utilized to automatically set (e.g., mechanically set) the position of the needle in a needle valve device. As a result, little or no interaction from a user is needed to calibrate a needle valve device upon start-up and/or after maintenance of the needle valve device.

Calibration collars or apparatus for automatically setting a position of a needle to a seat in a pressurized flow system including an actuator positioned to drive the needle relative to the seat are discussed in U.S. Provisional Application No. 61/607,930 and PCT/US2013/029580, and included herein. The actuator includes a shaft including an exterior liner and an interior extendable section. The calibration collar includes a housing, a first securing mechanism, a second securing mechanism, and a spring. The housing of the calibration collar includes a first end and a second end and the housing defines a channel sized to accept at least a portion of the shaft of the actuator. The first securing mechanism of the calibration collar is positioned at the second end of the housing and surrounds the channel. The first securing mechanism, when in a locked position, holds the housing to the exterior liner of the shaft. The second securing mechanism is independent of the first securing mechanism and is positioned between the first securing mechanism and the first end of the housing. The second securing mechanism, when in a closed position, grips at least a portion of an external perimeter surface of the interior extendable section of the shaft to clamp the housing to the shaft. The spring of the calibration collar is disposed at least partially in the first end of the housing and extends into the channel to apply a known load on the shaft when the shaft is seated in the housing.

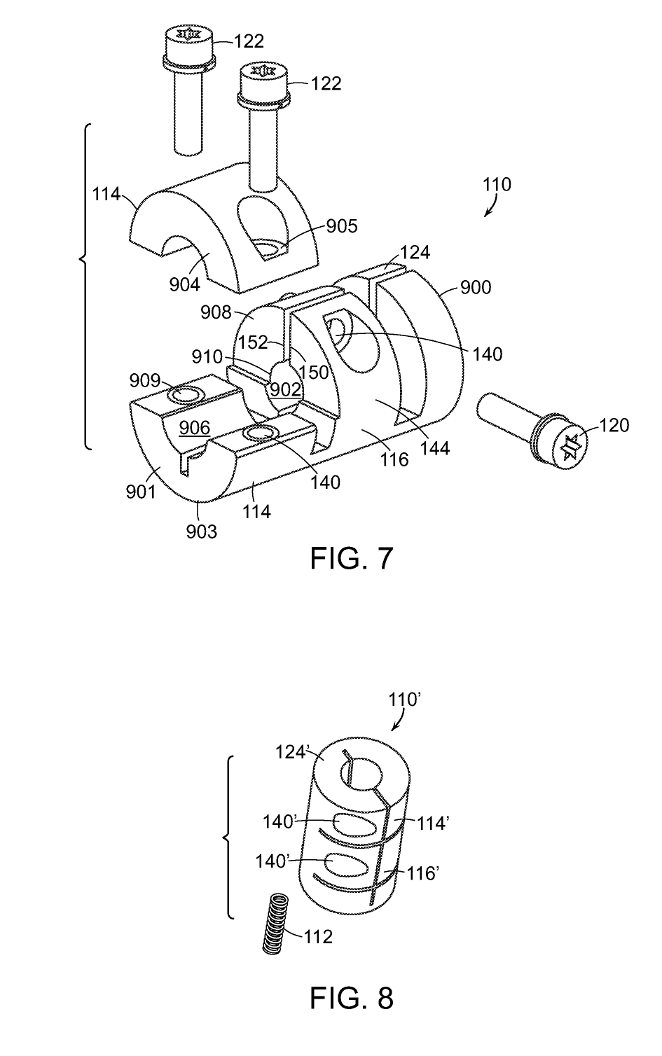

For example, FIG. 6 is a cross-sectional view of a dynamic pressure regulator 57 along a longitudinal axis L of the dynamic pressure regulator. The dynamic pressure regulator 57 can be implemented as a valve assembly that includes a proximal head portion 72, an intermediate body portion 74, and a distal actuator portion 76. The head portion 72 of the valve assembly can include an inlet 78 to receive the pressurized solvent stream and an outlet 80 through which the pressurized solvent stream is output such that the solvent stream flows through the head portion from the inlet 78 to the outlet 80. A seat 82 can be disposed within the head portion 72 and can include a bore 84 through which the solvent stream can flow from the inlet 78 to the outlet 80 of the head.

A needle 86 extends into the head portion 72 from the body portion 74 of the valve assembly through a seal 88. A position of the needle 86 can be controlled with respect to the seat 82 to selectively control a flow of the solvent stream from the inlet 78 to the outlet 80. In exemplary embodiments, the position of the needle 86 can be used to restrict the flow through the bore 84 of the seat 82 to increase the pressure of the CO₂-based chromatography system 10 and can selectively close the valve by fully engaging the seat 82 to interrupt the flow between the inlet 78 and the outlet 80. By controlling the flow of the solvent stream through the head portion based on the position of the needle 86, the pressure of the CO₂-based chromatography system 10 can be increased or decreased. For example, the pressure of the CO₂-based chromatography system 10 can generally increase as the needle 86 moves towards the seat 82 along the longitudinal axis L and can generally decrease as the needle 86 moves away from the seat 82 along the longitudinal axis L.

The actuator portion 76 can include an actuator 90, such as a solenoid, voice coil, and/or any other suitable electromechanical actuation device. In the present embodiment, the actuator 90 can be implemented using a solenoid having a main body 92 and a shaft 94. The shaft 94 can extend along the longitudinal axis L and can engage a distal end of the needle 86 such that the needle 86 and shaft can form a valve member. A position of the shaft 94 can be adjustable with respect to the main body 92 along the longitudinal axis L and can be controlled by a coil (not shown) of the main body 92, which generates a magnetic field that is proportional to an electric current passing through the coil and a load applied to the shaft. The electric current passing through the coil can be controlled in response to an actuator control signal received by the actuator 90. In some embodiments, the actuator control signal can be a pulse width modulated (PWM) signal and/or the actuator control signal can be determined, at least in part, by the feedback signal of the pressure transducer 59.

The position of the shaft 94 can be used to move the needle 86 towards or away from the seat 82 to increase or decrease pressure, respectively. In exemplary embodiments, a position of the shaft 94, and therefore a position of the needle 86 with respect to the seat 82 can be controlled and/or determined based on an amount of electric current flowing through the solenoid. For example, the greater the electrical current the closer to the needle 86 and shaft 94 are from the seat and the lower the pressure is in the CO₂-based chromatography system 10. The relationship between a position of the shaft 94 and the electric current flowing through the coil can be established through characterization of the actuator 90. The force imposed by the load on the solenoid can be proportional to the magnetic field. Similarly, the magnetic field can be proportional to the electric current flowing through the coil of the solenoid. For embodiments in which the actuator control signal is implemented as a PWM control signal, the pressure through the pressure regulator 57 (e.g., force balance between needle 86 and shaft 94) can be set by a correlation to the duty cycle of the PWM control signal, e.g., a percentage of the duty cycle corresponding to an “on” state.

The force imposed by the actuator 90 to set the pressure through the pressure regulator 57 can be manipulated for force control purposes by inclusion of a compressed spring 96. Spring 96 is compressed by collar 98 to apply a normalizing force to the actuator 90 through an exterior shaft liner 100. This normalizing force assists in providing a linear load force throughout the cycle of the actuator 90. In general, actuator 90 has a negative spring rate, such that shaft 94 when the actuator 90 is in an inactive state is forced in a direction opposite of outlet 80 (i.e., towards the end of the device labeled B), such that the force reduces as the solenoid stroke increases. To compensate for this force, compressed spring 96 applies a pressure to shaft 94 to counterbalance the negative spring rate of the actuator 90. In some embodiments, the spring rate selected for compressed spring 96 has a value that not only counterbalances but also applies a positive spring rate such that shaft 94 moves towards the end of the device labeled A.

To regulate pressure through device 57 from inlet 78 to outlet 80, the needle 86 and seat 82 are carefully positioned relative to one another. A calibrated position between the needle 86 and seat 82 is set at the position when the needle 86 first engages the bore 84 of the seat 82 to stop the flow of solvent. In general, care is taken to set this calibrated position, such that the needle 86 will not be jammed into the bore 84 during operation of pressure regulator 57. It is believed that prevention or at least minimization of the needle being jammed into the bore will extend the life of the pressure regulator and/or increase the working lifetime prior to a maintenance event.

During the lifetime of the pressure regulator 57, components, such as, for example the needle 86 or the seat 82 can become worn. These components may be replaced in maintenance events. After the maintenance event, the needle and seat need to be placed back into the calibration position.

Exemplary embodiments of the pressure regulator 57 include a calibration collar 110 secured to the shaft 94 to automatically (e.g., mechanically) reset the calibration position. That is, the calibration collar 110 applies a force on shaft 94 to lock further extension of the shaft 94. When the calibration collar 110 is secured onto shaft 94, a maintenance provider or user merely needs to position the shaft 94 in physical contact with the distal end of the needle and lock the calibration collar to mechanically set needle 86 relative to the seat 82 in the calibrated position.

To apply the force, the calibration collar 110 includes a spring 112 and two locking mechanisms 114 and 116. Locking mechanism 114 holds the calibration collar 110 to the exterior liner 100 of the shaft 94, whereas locking mechanism 116 grips the distal end 118 of the shaft 94 to clamp or lock the extended position of the shaft 94 to prevent jamming of the needle 86 into the seat 82. In the embodiment shown in FIG. 6, the locking mechanisms include fasteners 120 and 122 to secure a housing 124 forming the calibration collar 110 to the actuator 90.

During a maintenance event, the actuator 90 is inactivated (i.e., no signal is applied to drive the solenoid) and the flow of solvent is stopped. The needle 86 and seat 82 are in the calibrated position at the start of the maintenance event. That is, the needle 86 engages seat 82 to block bore 84. The calibration collar 110 attached to the shaft 94 as shown in FIG. 6 holds the needle and seat in this calibrated position. To obtain access to the distal end of the needle 86 and potentially to the seat, shaft 94 needs to be pulled back towards end B. In the calibration collar's configuration with both fasteners 120 and 122 secured, alignment of the needle 86, seat 82, and shaft 94 is maintained. However, to release this secured position, the user merely needs to loosen fastener 120 to release the grip of locking mechanism 116 from the distal end 118 of the shaft 94. The fastener 122 remains securely tightened or closed such that locking mechanism 114 continues to hold the housing 124 of the calibration collar 110 to the exterior liner 100. However, distal end 118 of the shaft 94 is free to move to allow access to the needle/seat for maintenance. At the conclusion of the maintenance event, the user places the proximal end 126 of the shaft 94 in contact with the needle 86 and tightens fastener 120 to reposition the needle 86 relative to the seat an in the calibrated position.

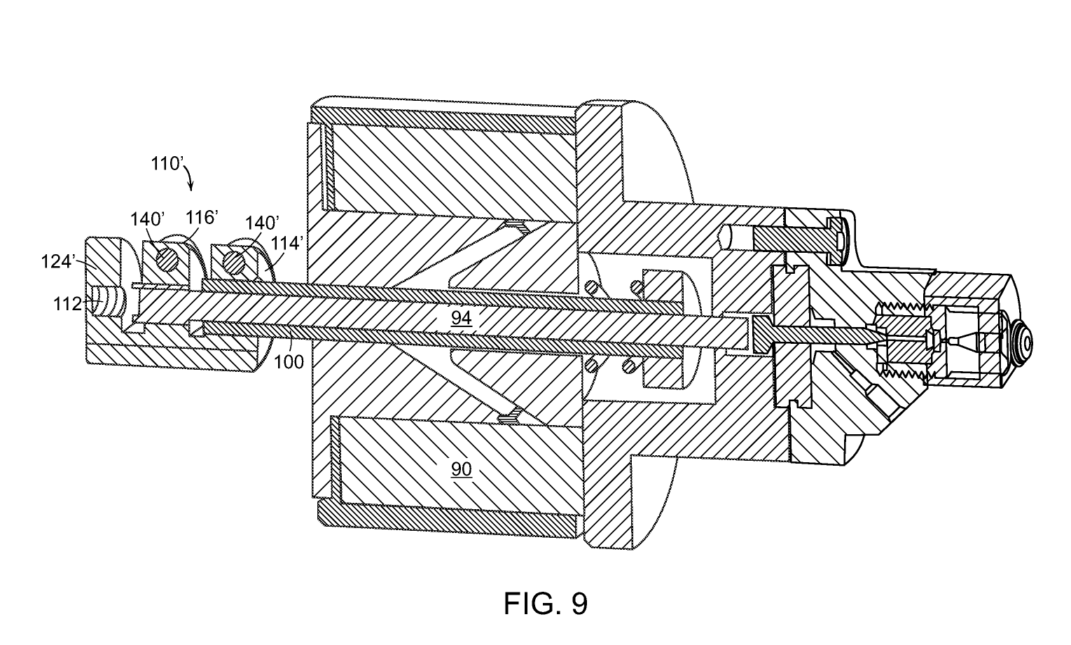

FIG. 7 is an exploded view of calibration collar 110. In FIG. 7, a portion of locking mechanism 114 (e.g., clamp 114) is shown in an unfastened state to show additional details of the interior of the calibration collar 110. The calibration collar 110 is formed from housing 124, typically manufactured from a metal, such as, for example, stainless steel or aluminum. The spring 112 (shown in FIG. 6 but not shown in FIG. 7) is disposed at least partially within a first end 900 of the housing. Locking mechanism 114 is disposed on the opposite end or the second end 901 and between locking mechanism 114 and the first end 900 is locking mechanism 116 (e.g., clamp 116).

A channel 902 is defined within housing 124 and the size of channel 902 is configured to accept at least a portion (such as, for example the distal end 118 and a portion of the exterior liner 100) of the shaft 94. The locking mechanism 114 surrounds channel 902 and is sized to receive the exterior liner 100. The locking mechanism 114 includes a base portion 903 and a top portion 904. When fasteners 122 are installed and tightened within openings 905, the locking mechanism is configured to secure base portion 903 to top portion 904 in a locked position, in which the housing 124 is held to the exterior liner 100. In embodiments, surface 906 defining a wall of the channel through locking mechanism 114 can be textured to apply a frictional force to further secure the calibration collar 110 to the actuator 90. Applied textures can include raised bumps, ribs, or grooves.

Locking mechanism 116 is also shown in an unfastened state in FIG. 7. Fastener 120 secures locking mechanism 116 in a closed position by forcing clamping portions 907 and 908 together at free ends 150 and 152. As shown in FIG. 7, each of the clamping portions 907 and 908 are integrally formed with the housing. In addition, base portion 903 of locking mechanism 114 is also integrally formed with the housing.

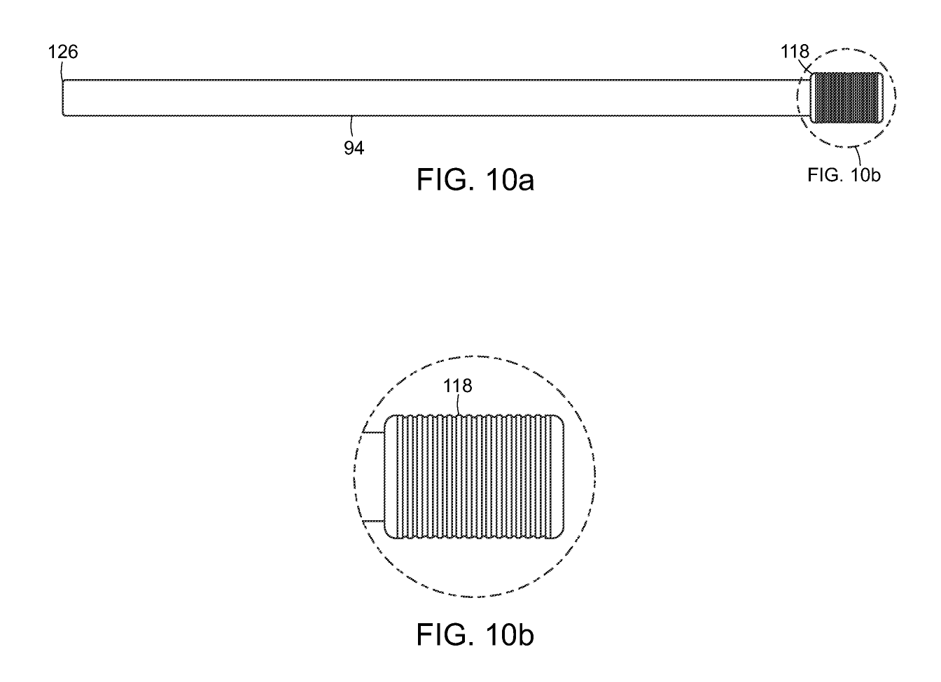

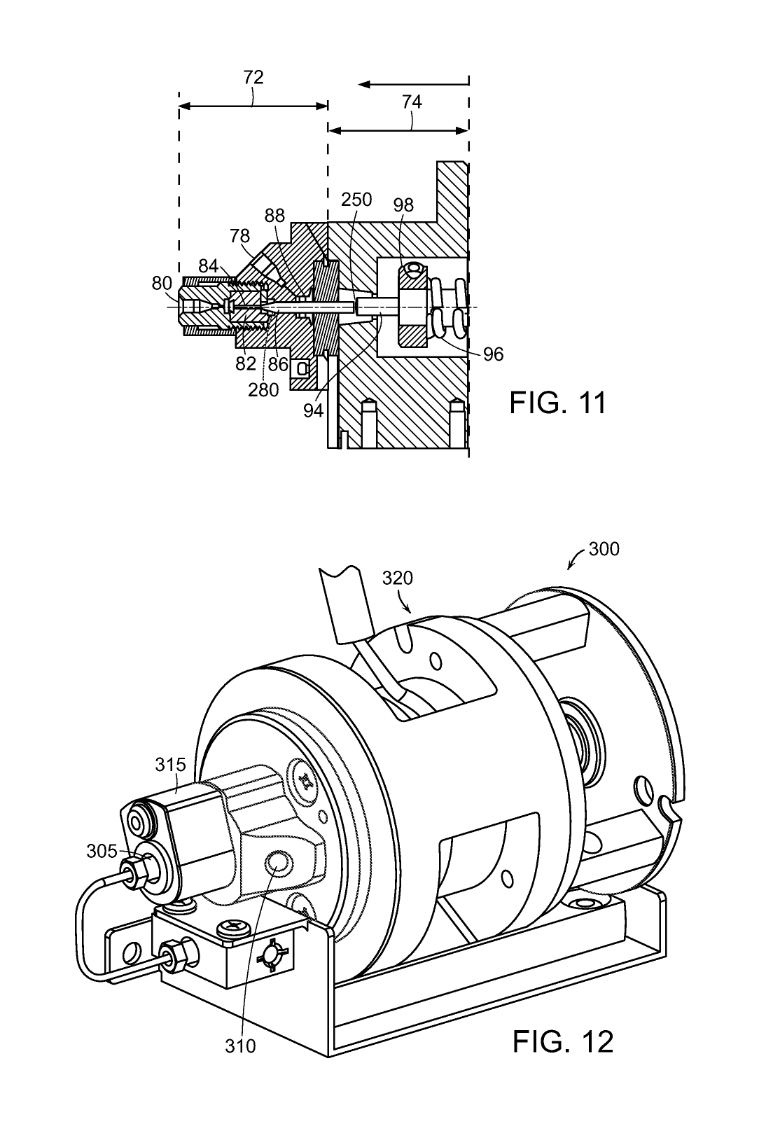

Locking mechanisms 114 and 116 can be implemented in numerous different configurations. For example, FIG. 8 shows an another calibration collar 110′ with locking mechanisms 114′ and 116′ each of which are integrally formed with housing 124′ and secured with a single fastener in each of openings 909. A cross-sectional view of calibration collar 110′ is shown in FIG. 9. In FIG. 9, calibration collar 110′ is secured to actuator 90 through shaft 94 and exterior liner 100.

In other embodiments, figures not shown, the locking mechanism 114 and/or 116 can be electromechanical locking assemblies in which an applied electric signal is used to open and close the mechanisms.

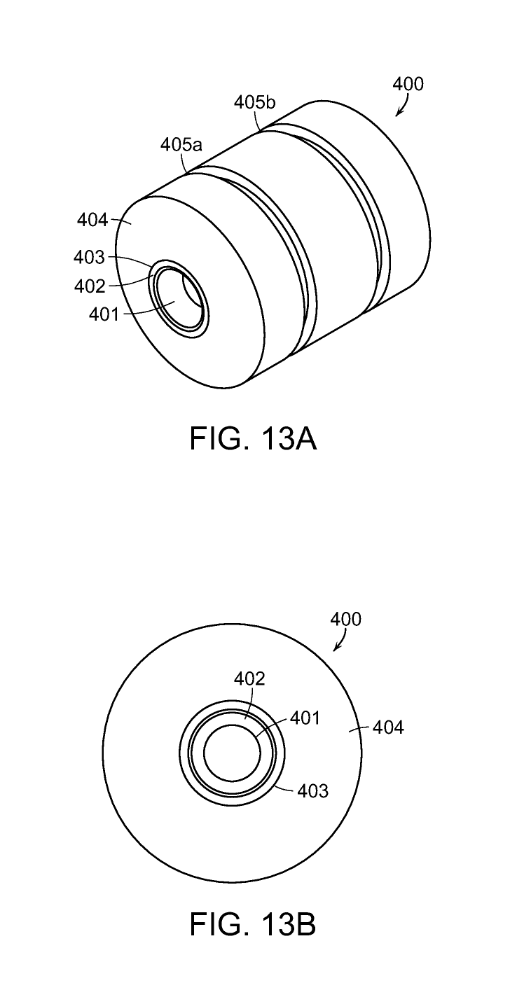

FIGS. 10a and 10b show an exemplary embodiment of shaft 94. As shown in FIGS. 6 and 9, shaft 94 lies within exterior liner 100 and is the portion of the actuator 90 that contacts needle 86. The proximal end 126 of shaft 94, when in use for pressure regulation, contacts the needle 86 to apply a force to the needle to change its position. In embodiments, the distal end 118 of the shaft 94 is secured within one of the exemplary calibration collars disclosed herein. The exterior surface of the distal end 118 can include a texture, such as the texture shown in FIGS. 10a and 10b to provide further grip or friction between locking mechanism 116 and the distal end 118. In addition to the exterior surface of the distal end 118 being textured, the interior surface 910 of a wall defining the channel 902 through locking mechanism 116 can also be textured. Applied textures can include raised bumps, ribs, grooves, or the like.

This mechanically self calibrating needle valve provides numerous advantages. For example, consistent needle calibration allows for consistent behavior, which ultimately provides better separation results in separation of one or more fatty acids. In addition to providing consistent calibration, the mechanically self calibrating needle valve provides increased efficiency and minimizes maintenance time. That is, the mechanically self calibrating needle valve provides an automatic or mechanically self-calibrating needle valve that simplifies maintenance events by limiting or eliminating user interaction (e.g., minimizes or eliminates decisions or calibration positioning by the user or controlling software) to recalibrate the position of the needle relative to the seat in the field after maintenance events.

While the foregoing general describes the mechanically self calibrating needle valve, variations and other methods of this component are as described in U.S. Provisional Application No. 61/607,930 and the needle relative to the seat are discussed in U.S. Provisional Application No. 61/607,930 and PCT/US2013/029580.

### Force Balance Needle Valve Pressure Regulator

As described in U.S. Provisional Application No. 61/607,935 and PCT/US2013/029543, the contents of which are incorporated herein by reference, the dynamic back pressure regulator and force balance needle as used in the present disclosure of the CO₂-based chromatography system 10, minimizes flow or compositional changes of the mobile phase when separating one or more fatty acids. Thus, exemplary embodiments of the CO₂-based chromatography system 10 comprise a dynamic back pressure regulator and a force balance needle between the drive mechanism and the system pressure. Such assemblies and methods can dampen the effects caused by pressure drops or pressure-related inconsistencies that may occur during introduction of a mobile phase, and/or throughout the pressure regulation of a mobile phase in the CO₂-based chromatography system 10.

For example, exemplary embodiments comprise a needle valve driven by a solenoid or other type of actuator. Generally, the assemblies and methods include, for example, determining the optimal position of a needle with a regulator, such that minor differences or pressure fluctuations occurring from the combination of the internal pressure of a solenoid and the internal pressure created from the introduction of a mobile phase, are counterbalanced or compensated for by the needle. As presented herein, the needle valve and solenoid are designed for enhanced stability and have a minimal change in force through the operating stroke (e.g., approximately 0.010 of an inches). The current to the solenoid controls the force the solenoid applies to the needle and the pressure area on the needle provides a counter force to the solenoid assembly. In certain instances, the needle naturally finds a position such that the pressure force and the solenoid force balance, such that the pressure can be directly set by commanding a force out of the solenoid to give the desired pressure.

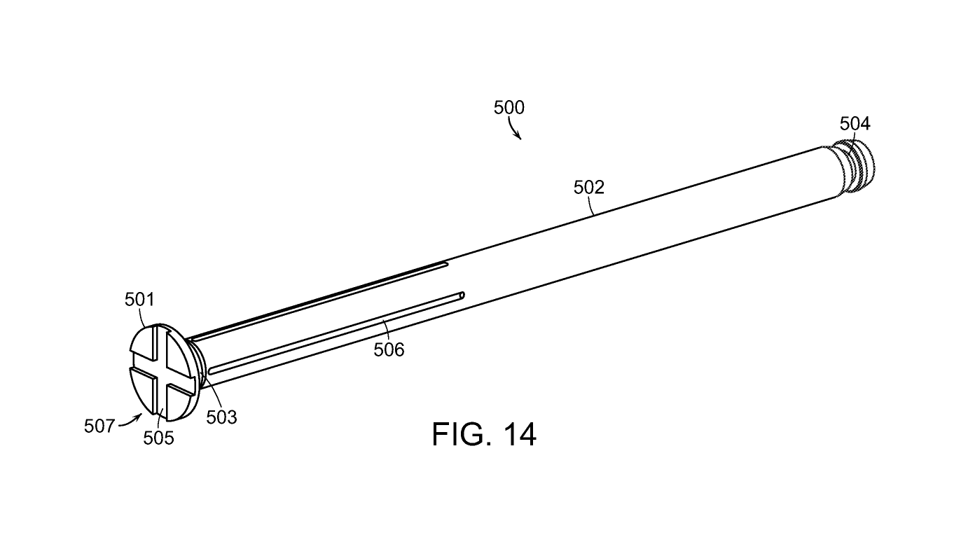

The pressure of system 10 is dynamically regulated in the back pressure regulator 57. FIG. 11 illustrates and embodiment of the proximal head portion 72 as described above and shown in FIG. 6. According to an embodiment of the present disclosure, a mobile phase, such as CO₂ enters the head portion through inlet 78, thereby creating a first pressure in the head portion 72. The actuator (e.g., a force balanced solenoid, such as a commercially available solenoid modified with compression spring 96 shown in FIG. 6 or a voice coil) applies a constant force through shaft 94 to the back portion of the needle 250 needle 86 such that the needle is set to an appropriate location with respect to seat 82 to create the pressure entered through controller 102.

Due to the increase pressure build up in the head portion, a second pressure is created on the head portion of the needle 280. Upon disruptions, pressure drops, or pressure increases from the gas entering thorough inlet 78, a pressure differential occurs in the head portion, thereby generating third pressure in the head portion. Once the third pressure is created needle 86, independent of the constant force applied by the actuator 76, moves either further forward into seat 82 (i.e. towards outlet 84) or relaxes back (i.e. towards shaft 94) to maintain in close proximity to the actuator. The movement of the needle 86 due to the third pressure is relatively small (e.g., from about 0.001 to about 0.05 inches). That is, the needle moves to compensate for pressure differentials between the second and third created pressures and occurs without adjusting or controlling the force created by the actuator 90.

When discussed from a force balance perspective, there is a balance between the force applied to the back of the needle 250 by the actuator and the force applied to the head portion of the needle 280 by the pressure coming from inlet 78. Upon disruptions, pressure drops, or pressure increases from the media entering thorough inlet 78, the forces become unbalanced. This is primarily controlled by the restriction created by the needle 86 to seat 82 gap. If the pressure rises, the force on the end of the needle increases, pushing it away from the seat; therefore reducing the restriction until the pressure created by the restriction is once again equal to the actuator force. If the pressure reduces, the force on the end of the needle decreases, and the actuator pushes it towards the seat; therefore increasing the restriction until the pressure created by the restriction is once again equal to the actuator force.

The force balance needle valve pressure regulator provides numerous advantages. For example, by incorporating the force balance needle valve pressure regulator, pressure changes associated with a change in solvent or a change in flow are minimal. As a result, pressure is only affected by any slope in the force vs. stroke of the solenoid. In addition, the controller described herein requires little movement to accommodate a change in condition. That is, a given current provides a specific back pressure that varies only by tolerances of the actuator. As a result, a high degree of control can be achieved. Further, the force balance approach cancels pressure changes due to flow or composition fluctuations. Thus, the use of the force balance needle valve pressure regulator provides better pressure control over changing conditions when separating one or more fatty acids.

While the foregoing general describes the force balance needle valve pressure regulator, variations and other methods of this component are as described in U.S. Provisional Application No. 61/607,935 and PCT/US2013/029543.

### Low Volume, Pressure Assisted, Stem and Seat Vent Valve

As is known in the art, it is generally desirable to have the ability to vent a CO₂-based chromatography system when it is not in use. However, if the vent valve significantly adds to the system volume, the ability of the back pressure regulator to control pressure in a CO₂-based chromatography system can be compromised. Vent valves are generally configured to push the needle into the seat to stop flow through the vent valve. In this configuration, a pressure assist can be implemented to open the vent valve. However, the seal of the needle against the seat and/or the bore inside the seat add to the exposed close volume of the vent valve. An increased pressure assist ensures the valve seals properly at higher pressures where non-pressure assisted valves tend to leak. The exposed volume of the vent valve requires the CO₂-based chromatography system to compress a larger volume to increase pressure. In particular, the maximum rate of pressurization is directly related to the solvent stiffness times the flow rate divided by the system volume. Increased volume thereby decreases the response of the CO₂-based chromatography system and leads to more lag and/or slower control attributes.

As used herein, and as described in U.S. Provisional Application No. 61/607,956 and PCT/US2013/029529, the contents of which are incorporated herein by reference, vent valves that minimize the exposed volume of the valve body and/or implement a system pressure to assist in sealing the vent valve are provided. Such vent valves comprise a valve body that includes a seat retainer, a needle and a seat. The seat includes a bore extending therethrough and the needle includes a needle stem and a needle head. In particular, the seat is disposed inside the seat retainer and the needle stem is disposed inside the bore. The needle can be configured to be pulled through the seat to stop flow through the bore. Conversely, the needle can be configured to be pushed through the seat to start flow through the bore.

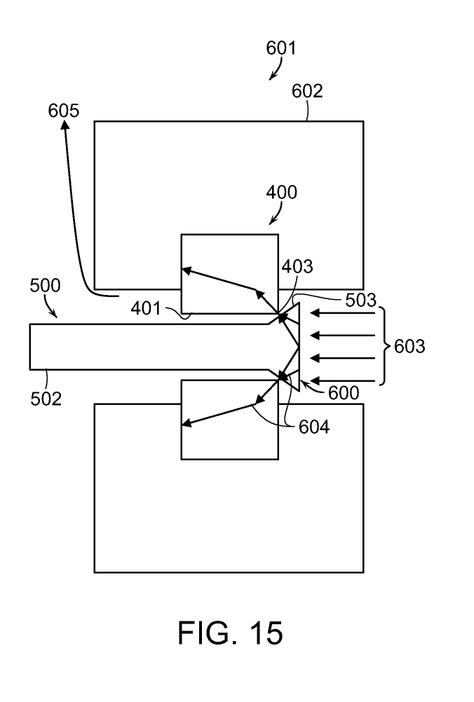

With reference to FIG. 12, an exemplary vent valve 300 is depicted, including a valve body, a pressurized inlet port 305 and an outlet port 310. The vent valve 300 can have two sections, i.e., a vent valve actuator section 320 and a vent valve head section 315. The vent valve head section 315 includes the seat retainer, needle and seat to be implemented in the exemplary vent valve 300. It should be understood that the dimensions and/or configurations of the vent valve 300 are merely exemplary and other embodiments can have different dimensions and/or configurations.

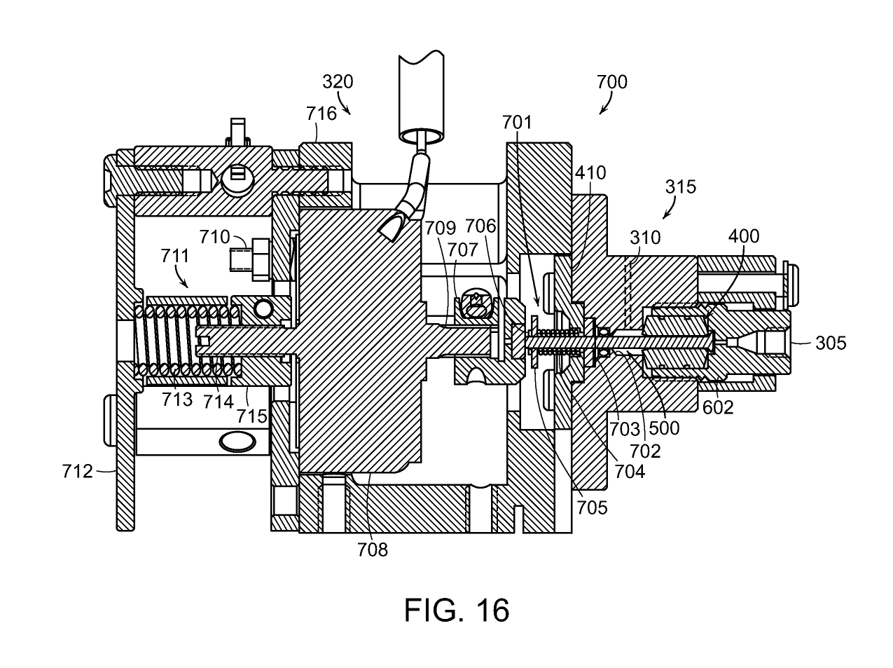

Turning to FIGS. 13(a) and (b), an exemplary seat 400 is illustrated, including a bore 401 extending therethrough. The bore 401 is greater in diameter than a needle stem diameter to ensure the needle stem can pass through unimpeded. It should therefore be understood that the bore 401 dimension can differ based on the needle stem being implemented. The bore 401 can include a chamfered outlet 402, e.g., angled, beveled, outwardly sloping, and the like, to create a larger opening surface area than the bore 401 diameter for sealing against the needle head. For example, the chamfered outlet 402 can be at about, e.g., 15°, 20°, 25°, 30°, 35°, 40°, 45°, and the like. In other embodiments, the chamfered outlet 402 can be at an angle less than the taper of the angled sealing surface of the needle. For example, the chamfered outlet 402 angle can be half or less of the angle of the taper of the angled sealing surface of the needle. The larger opening surface area created by the chamfered outlet 402 can assist in centering and/or guiding the needle head as it is pulled into the bore 401. The edge adjoining the chamfered outlet 402 of the bore 401 and outer side surfaces 404 of the seat 400 can be defined by the bore edge 403.

The seat 400 may include circumferential seat grooves 405a and 405b to enhance the fastening of the seat 400 inside the seat retainer. In particular, an inner surface of the seat retainer can include protrusions, e.g., spikes, ridges, and the like, configured and dimensioned to mate with the seat grooves 405a and 405b. Thus, as the seat retainer is fastened and/or tightened around the seat 400 and/or the seat 400 is pressed into the seat retainer, the seat retainer protrusions can mate with the seat grooves 405a and 405b to prevent undesired motion of the seat 400 within the seat retainer. Although illustrated with two seat grooves 405a and 405b, other embodiments of the exemplary seat 400 can have less and/or more seat grooves, e.g., zero, one, two, three, four, five, and the like.

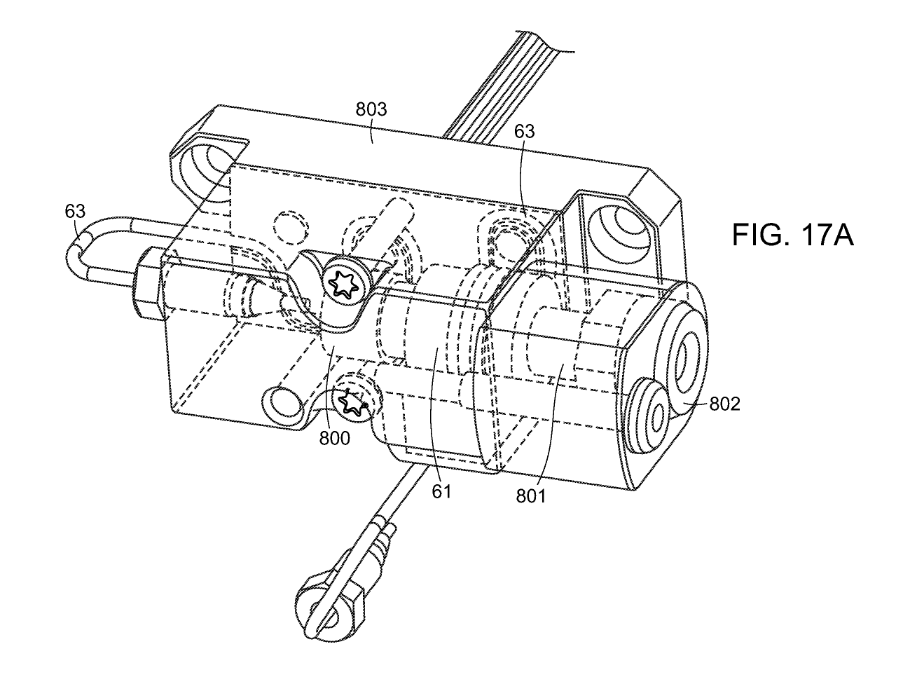

Turning now to FIG. 14, an exemplary needle 500 is illustrated, including a needle head 501 and a needle stem 502. The diameter of the needle head 501 is greater than the diameter of the needle stem 502 to provide a durable and/or tight seal between the needle head 501 and the seat 400 when the needle stem 502 is pulled through the bore 401. The diameter of the needle stem 502 can be configured and dimensioned to pass unimpeded through the bore 401. In particular, the diameter of the needle stem 502 can be slightly smaller than the diameter of the bore 401 to permit the needle stem 502 to pass through the bore 401, while supporting the needle 500. Thus, no matter which dimensions and/or configurations of the needle 500 and/or seat 400 are being implemented, the diameter of the needle stem 502 will always be slightly smaller than the diameter of the bore 401.

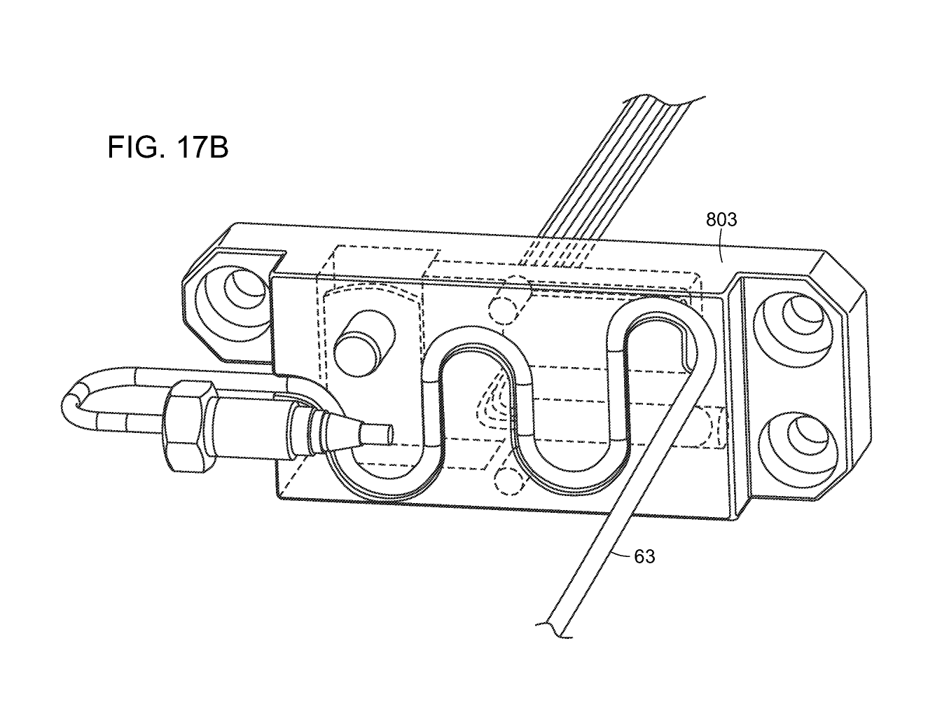

Turning now to FIG. 15, a seat retainer assembly 601 is depicted, including a seat retainer 602, a seat 400 and a needle 500. Although referring to a needle 500, it should be understood that the exemplary seat retainer assembly 601 can instead include a needle 500. The seat retainer 602 can be securely disposed inside the vent valve head section 315 of FIG. 12. The seat 400 can be securely disposed inside the seat retainer 602. As described above, although not illustrated in FIG. 15, the seat grooves 405a and 405b can mate with protrusions, e.g., ridges, spikes, or the like, of the internal contact surface of the seat retainer 602 to prevent undesired movement of the seat 400 in the seat retainer 602. The needle stem 502 is at least partially disposed inside the bore 401 of the seat 400 and can be translated within the bore 401. The keeper groove 605 at the distal end of the needle stem 502 can be secured to a stem return spring mechanism (not shown).

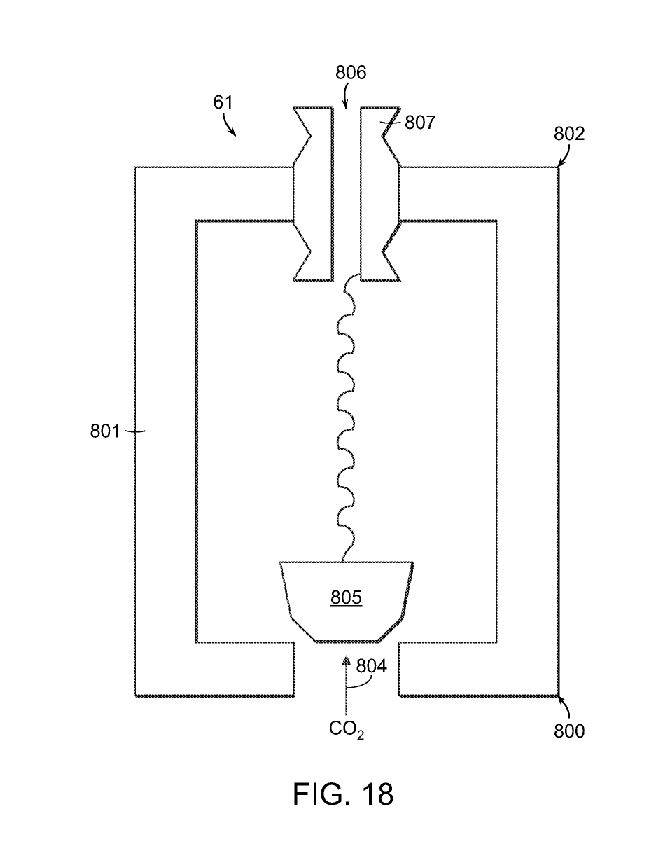

Turning now to FIG. 16, an exemplary embodiment of a vent valve 700, e.g., a solenoid valve, is depicted in an open position, i.e., a flow path exists between the angular sealing surface 503 of the needle 500 and the bore edge 403 of the seat 400. The vent valve 700 includes a valve body 64, which includes a vent valve actuator section 320 and a vent valve head section 315. The seat retainer assembly 300 is securely disposed inside the vent valve head section 315, including the seat retainer 302, the seat 400 and the needle 500. The vent valve head section 315 further includes the inlet port 305 and the outlet port 310.

While the foregoing general describes the low volume, pressure assisted, stem and seat valve, variations and other methods of this component are as described in U.S. Provisional Application No. 61/607,956 and PCT/US2013/029529.

### Combination Dynamic and Static Pressure Regulator

Embodiments include setting the static pressure regulator inlet pressure to a pressure above the critical pressure for the mobile phase media. As a result, the mobile phase media passing through the dynamic pressure regulator is maintained in the liquid phase. In general, a dynamic pressure regulator, can better (e.g., more consistently) control pressure of a single phase (e.g., liquid phase) material across its inlet and outlet. In addition, controlling the phase change of the mobile phase media within the static pressure regulator and heating at least a portion of the static pressure regulator to prevent or minimize freezing and its effects on the static pressure regulator. The combination of regulators referenced herein, and as described in U.S. Provisional Application No. 61/607,924 and PCT/US2013/029524, the contents of which are incorporated herein by reference, can dampen damaging effects caused by pressure drops of a supercritical or near supercritical fluid, while providing accurate pressure control.

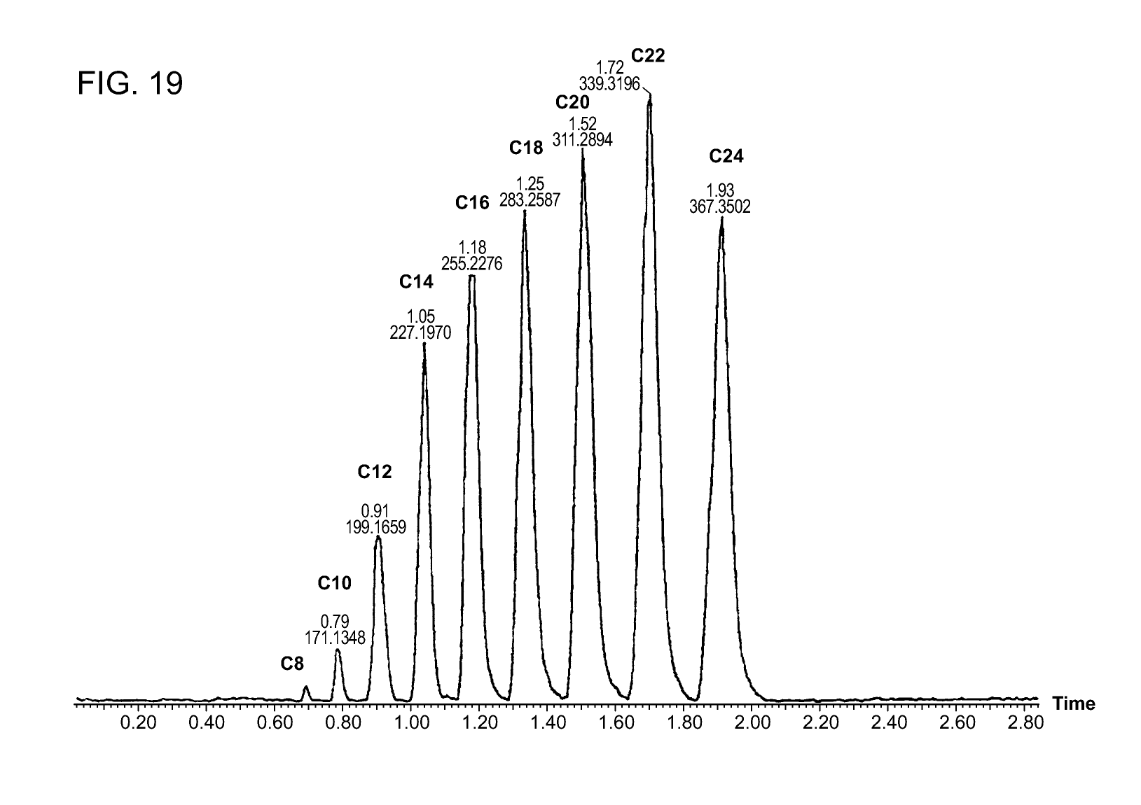

The method includes, for example, pre-heating and/or post-heating the mobile phase to eliminate issues related to, e.g., condensation, frost, clogging and sputtering, and pressure disturbances and fluctuations throughout the pressurized flow system. For example, FIGS. 17a and 17b illustrate an embodiment in which the heating element 63 extends from a location prior to (e.g., upstream of) a first end 800 of static regulator 61 and continues along a body 801 of the static regulator 61. In the embodiment shown in FIGS. 17a and 17b (FIG. 17b showing a similar view to that of FIG. 17a, but with the static pressure regulator 61 removed), heating element 63 is a coil or serpentine tube which is heated to a temperature sufficient to keep the mobile phase above a temperature of about 0° C. For example, the heating element 63 can supply enough thermal energy to prevent or minimize the effects of freezing within the static pressure regulator 61. To heat the static pressure regulator, the heating element 63 is placed in thermal contact with the static pressure regulator 61. To position and hold heating element 63 in contact with static pressure regulator, block 803 secures the heating element 63 and static regulator 61 together. In some embodiments (not shown), the heating element 63 can extend pass a second end 802 of the static pressure regulator 61. Some embodiments include more than one (e.g., two, three, four) heating elements 63 to heat the static pressure regulator 61.

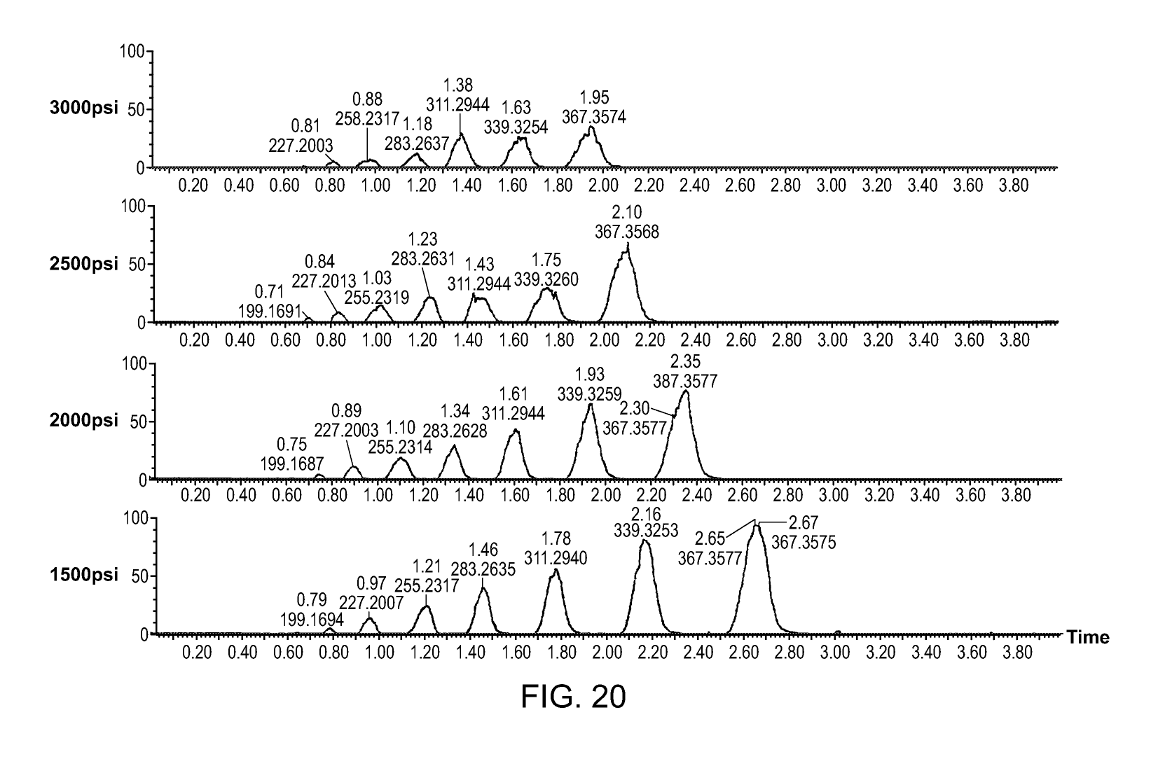

Referring to FIG. 18, static pressure regulator 61 is a passive pressure regulator. That is, the pressure of the static pressure regulator 61 is set and does not change during an operative run of system 10. In embodiments, the pressure at inlet 804 is set above the critical pressure of the mobile phase media. For example, in some embodiments, the pressure at inlet 804 is set to a pressure falling within a range of about 1500 to 1070 psi. In other embodiments, the pressure is set within a range of about 1400 to 1150 psi, for example, 1250 psi.

To set the pressure of static pressure regulator 61, the static pressure regulator 61 is fitted with screw 807. In an exemplary embodiment, the mobile phase, such as CO2, passes through inlet 804 and pushes on poppet 805. Screw 807 is adjusted to set the desired pressure to attain constant pressure on poppet/coil 805. The mobile phase move around the poppet 805 and exits through a hole 806 in the center of the screw 807. Other exit paths, in addition to, or alternative of hole 806 are possible.

By setting the pressure of the static pressure regulator 61 to a pressure above the critical pressure, the mobile phase media is maintained in a single phase (e.g., liquid phase) in the dynamic pressure regulator 57. As a result, the dynamic pressure regulator is not exposed to a phase change, nor is it exposed to a dual phase or multiphase (e.g., combination of liquid and gas phase) material. In general, dynamic pressure regulators can more consistently control the pressure of a single phase material (e.g., a material having a substantially constant density). Therefore, by maintaining the phase of the mobile phase media as a liquid throughout the dynamic pressure regulator, improvements in pressure control can be achieved.

The combined static and dynamic pressure regulator of the present disclosure, and as described in U.S. Provisional Application No. 61/607,924 and PCT/US2013/029524 provides numerous advantages. For example, the static and dynamic pressure regulator of the present disclosure can control pressure while minimizing damaging effects of phase changes and pressure drops. In general, the inlet to the static pressure regulator can be set at a pressure above the critical pressure for the mobile phase material, thereby guaranteeing that the mobile phase material is in a liquid phase through the dynamic pressure regulator. As a result of being a liquid phase throughout its flow path within the dynamic pressure regulator, pressure can be consistently controlled. Changes in phase can cause the mobile phase to gasify upstream of the regulator causing pressure consistency problems. Thus, by forcing the phase of the mobile phase media to remain as a liquid throughout the dynamic pressure regulator, improvements in pressure control consistency can be achieved.

Another advantage provided by static and dynamic pressure regulator of the present disclosure includes a reduction in damaging effects caused by pressure drops of a supercritical or near supercritical fluid. For example, by restricting the phase change of the mobile phase media to occur in the static pressure regulator, one can localize the effects of the phase change. In general, the phase change of CO₂ from a liquid to a supercritical fluid is endothermic, and thus the phase change location needs to be heated to prevent freezing. By controlling the location of the phase change (e.g., restricting phase change to the static pressure regulator), heating can be simplified and localized to this particular location (e.g., static pressure regulator). In addition, in the event that the localized heating does not prevent all damage, the damage is limited to the static pressure regulator. As a result, only the static pressure regulator component, and not the dynamic pressure regulator, would be repaired or replaced.

While the foregoing general describes the low volume, pressure assisted, stem and seat valve, variations and other methods of this component are as described in U.S. Provisional Application No. 61/607,924 and PCT/US2013/029524. Also, additional methods and advantages of various components of the CO₂-based chromatography systems used in the present disclosure are provided in U.S. Provisional Application No. 61/607,919 (“Device Capable of Pressurization and Associated Systems and Methods”) and PCT/US2013/029556; U.S. Provisional Application No. 61/607,952 (“Modular Solenoid Valve Kits and Associated Methods”) and PCT/US2013/029561; U.S. Provisional Application No. 61/607,913 (“Limiting a Rate of Pressurization in a Pressurized Flow System having a Configurable System Volume”) and PCT/US2013/029536; U.S. Provisional Application No. 61/607,910 (“Pressure Related Hysteresis Manipulation in a Pressurized Flow System”) and PCT/US2013/029539; U.S. Provisional Application No. 61/607,943 (“System and Method for Minimization of Force Variation in a Solenoid within a Pressurized Flow System”) and PCT/US2013/029531; and U.S. Provisional Application No. 61/695,838 (“Method for Improving the Separation Efficiency in Supercritical Fluid Chromatography”) and PCT/US2013/057507. The entire teachings of these applications are incorporated by reference herein.

In some embodiments, the system pressure of the CO₂-based chromatography system described herein, which is the pressure of the liquid as it exits the pump, is from about 1000 to about 9000 psi, e.g., from about 1500 psi to about 3000 psi. In some embodiments, the system pressure controller of the CO₂-based chromatography system provides and maintains steady pressure levels, and provides accurate and reproducible pressure gradients while maintain or producing CO₂ at or near supercritical state.

In an embodiment, the backpressure regulator of the CO₂-based chromatography system provides steady pressure levels and improved pressure gradients. In some embodiments, the pressure at the exit of the system, as controlled by the backpressure regulator is from about 1000 psi to 9000 psi. In some embodiments, the pressure is from about 1000 to about 3000 psi. In other embodiment, the pressure is about 1885 psi.

In an exemplary embodiment, the present disclosure provides a method of separating one or more fatty acids, comprising placing a sample (e.g., a biological sample or commercial sample) in a CO₂-based chromatography system comprising a chromatography column and eluting the sample by a gradient of organic solvent and a mobile phase fluid comprising CO₂ to substantially resolve the one or more fatty acids, wherein the CO₂-based chromatography system comprises: a chromatography column; an operating system pressure of about 1000 to about 9000 psi and a backpressure of about 1000 to about 9000 psi (e.g., about 1500 to 3000 psi); and one or more pumps for delivering a flow of the mobile phase fluid comprising CO₂.

In an exemplary embodiment, the CO₂-based chromatography system further comprise an injection valve subsystem in fluidic communication with the one or more pumps and the chromatography column. In another exemplary embodiment, the injection valve system comprises an auxiliary valve and an inject valve. The auxiliary valve may comprise 1) an auxiliary valve stator, comprising a first plurality of stator ports, in fluidic communication with the one or more pumps and the chromatography column and 2) an auxiliary valve rotor comprising a first plurality of grooves. The inject valve may comprise 3) an inject valve stator comprising a second plurality of stator ports and 4) an inject valve rotor comprising a second plurality of grooves.

In an exemplary embodiment, the CO₂-based chromatography system further comprise 5) a sample loop fluidically connected to the inject valve stator for receiving a sample slug to be introduced into a mobile phase fluid flow and 6) fluidic tubing fluidically connecting the auxiliary valve stator and the inject valve stator. The auxiliary valve rotor may be rotatable, relative to the auxiliary valve stator, between a plurality of discrete positions to form different fluidic passageways within the auxiliary valve. The inject valve rotor may be rotatable, relative to the inject valve stator, between a plurality of discrete positions to form different fluidic passageways within the inject valve. The respective positions of the auxiliary valve rotor and the inject valve rotor may be coordinated in such a manner as to allow the sample loop and the fluidic tubing to be pressurized to a high system pressure with the mobile phase fluid before they are placed in fluidic communication with the chromatography column.

In some embodiments, the volume of sample needed to be injected to the SFC system of the subject technology is from about 0.10 μL to 20 μL. However, those of skill in the art appreciate that the volume of sample to be injected depends primarily on the concentration of the analytes in that sample and also on what type of detection method being used. For example, if MS (Mass Spectroscopy) is the detection method used in tandem with the CO₂-based chromatography system, smaller injection volumes are typically required. In some embodiments, the CO₂-based chromatography system when in tandem with an MS/MS can facilitate detection of analytes in picogram (pg, one trillionth (10⁻¹²) of a gram) ranges.

In some embodiments, the temperature fluctuations in the pumping systems which may result in system pressure fluctuations are reduced or eliminated, which leads to a reduced baseline noise of chromatograms of the CO₂-based chromatography system.

In some embodiments, the CO₂-based chromatography system minimizes the consumption of mobile phase solvents (e.g. methanol) thereby generating less waste for disposal and reducing the cost of analysis (by more than 100 fold, in some cases) per sample.

### Column Chemistry

Given the above advantages of the components and methods of the present CO₂-based chromatography systems and techniques, the solid stationary phase of the column can comprise smaller mean particles sizes, e.g., within the range of 0.1-3 microns, though a smaller or larger size could be selected if appropriate for a desired application. In various examples, the mean particle size is 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9 or 3.0 microns.

In general, particle size can be selected in view of the desired pressure and/or flow rate. However, in general, smaller particle sizes allow for higher flow rates and higher efficiency, which yield faster, more sensitive separations. As such, in an exemplary embodiment, the chromatography columns described herein comprise reverse or normal phase silica-based particles (e.g., high strength silica particles) having an average particle size of about 1.8 microns and optionally comprising one or more diol ligands. Particles suitable for the technology disclosed herein include e.g., high strength silica particles, that is, 100% silica particles for use in applications up to 15,000 psi [1034 bar]. A suitable commercially available column that includes such particles is, e.g., the ACQUITY UPC² HSS C18 SB column, Waters Corporation, Milford Mass. Other particles that are suitable for the technology disclosed herein further include e.g., ethylene bridged hybrid particles having an average particle size of about 1.7 microns, examples of which are described in U.S. Pat. No. 6,686,035. One such commercially available column that include such particles is, e.g., the ACQUITY UPC² BEH C18 SB column, Waters Corporation, Milford Mass. In an embodiment, the total elution times for the one or more fatty acids is less than about 5 minutes (e.g., less than about 3 minutes) on a chromatography column having a length of about 100 mm. In another embodiment, the retention times of the one or more fatty acids range from about 0.5 to about 2 minutes.

The solid stationary phase can include pores having a mean pore volume within the range of 0.1-2.5 cm/g. In various examples, the mean pore volume is 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, or 2.5 cm/g. In some embodiments, porous particles may be advantageous for more biologically based lipid samples because they provide a relatively large surface area (per unit mass or column volume) for protein coverage and at the same time as the ability to withstand high pressure. Solid stationary phases can include pores having a mean pore diameter within the range of 100-1000 Angstroms. For example, the mean pore diameter can be about 100, 200, 300, 400, 500, 600, 700, 800, 900, 1000, or any value or range therebetween.

In certain embodiments, said chromatographic column, includes (a) a column having a cylindrical interior for accepting a packing material, and (b) a packed chromatographic bed comprising a porous material comprising an organosiloxane/SiO₂ material having the formula SiO₂/(R²ₚR⁴_{q}SiOₜ)ₙ or SiO₂/[R⁶(R²ᵣSiOₜ)ₘ]ₙ, (disclosed in U.S. Pat. Nos. 7,919,177; 7,223,473, and 6,686,035, each of which is hereby incorporated herein by reference) wherein R² and R⁴ are independently C₁-C₁₈ aliphatic, styryl, vinyl, propanol, or aromatic moieties, R⁶ is a substituted or unsubstituted C₁-C₁₈ alkylene, alkenylene, alkynylene or arylene moiety bridging two or more silicon atoms, p and q are 0, 1 or 2, provided that p+q=1 or 2, and that when p+q=1, t=1.5, and when p+q=2, t=1; r is 0 or 1, provided that when r=0, t=1.5, and when r=1, t=1; m is an integer greater than or equal to 2, and n is a number from 0.03 to 1. In an embodiment, the material has been surface modified. In another embodiment, the material has been surface modified by a surface modifier selected from the group consisting of an organic group surface modifier, a silanol group surface modifier, a polymeric coating surface modifier, and combinations thereof. In another embodiment, the surface modifier has the formula Za(R′)bSi—R, where Z═Cl, Br, I, C₁-C₅ alkoxy, dialkylamino or trifluoromethanesulfonate; a and b are each an integer from 0 to 3 provided that a+b=3; R′ is a C₁-C₆ straight, cyclic or branched alkyl group, and R is a functionalizing group.

The functionalizing group R may include alkyl, alkenyl, alkynyl, aryl, cyano, amino, diol, nitro, cation or anion exchange groups, or alkyl or aryl groups with embedded polar functionalities. Examples of suitable R functionalizing groups include C₁-C₃₀ alkyl, including C₁-C₂₀, such as octyl (C₈), octadecyl (C₁₈), and triacontyl (C₃₀); alkaryl, e.g., C₁-C₄-phenyl; cyanoalkyl groups, e.g., cyanopropyl; diol groups, e.g., propyldiol; amino groups, e.g., aminopropyl; and alkyl or aryl groups with embedded polar functionalities, e.g., carbamate functionalities such as disclosed in U.S. Pat. No. 5,374,755, the text of which is incorporated herein by reference. In an embodiment, the surface modifier may be an organotrihalosilane, such as octyltrichlorosilane or octadecyltrichlorosilane. In an embodiment, the surface modifier may be a halopolyorganosilane, such as octyldimethylchlorosilane or octadecyldimethylchlorosilane. In an exemplary embodiment, the chromatography columns described herein comprise reverse or normal phase silica-based particles (e.g., high strength silica particles) having an average particle size of about 1.7 microns and optionally comprising one or more diol ligands.

In some embodiments, depending on the complexity and nature of the sample fatty acids, the separation is accomplished using high strength silica particles as the stationary phase optionally modified with an alternate ligand (polar, non-polar, or ionic, such as e.g., diol coated), or with no additional surface modification at all. Technologies surrounding such particles relative to the present disclosure can be found in e.g., U.S. Provisional application Ser. No. 13/366,009 (Methods and Materials for Performing Hydrophobic Interaction Chromatography), filed Dec. 23, 2011, and U.S. application Ser. No. 13/580,884 (Methods, Compositions, Devices, and Kits for Performing Phospholipid Separation), filed Aug. 23, 2012, both of which are incorporated herein by reference, and in U.S. Pat. No. 6,686,035 (Porous Inorganic/Organic Hybrid Particles for Chromatographic Separations and Process for Their Preparation); U.S. Pat. No. 7,223,473 (Porous Inorganic/Organic Hybrid Particles for Chromatographic Separations and Process for Their Preparation); and U.S. Pat. No. 7,919,177 (Porous Inorganic/Organic Hybrid Particles for Chromatographic Separations and Process for Their Preparation), each of which is incorporated herein by reference. Also, as described herein by way of example, a suitable commercially available column that includes such particles is, e.g., the ACQUITY UPC² HSS C18 SB column, Waters Corporation, Milford Mass. Additionally, the separation could be achieved on various particles sizes below 5 mm in diameter. In some embodiments, the column internal diameter (ID) is between about 2 mm to 3 mm, while the column length is between about 30 mm to 150 mm. In one embodiment, the internal diameter is about 3 mm and the column length is about 100 mm.

In an embodiment herein, the chromatography column comprises an internal diameter of less than about 3.5 (e.g., internal diameters of about 3.0 or about 2.1), a length of less than about 175 mm (e.g., a length of about 150 mm or about 100 mm), and the high strength silica particles described above having an average particle size of about 1.7 microns and optionally comprising one or more diol ligands. Alternatively, the particles described for the chromatography columns herein comprise ethylene bridged hybrid particles having an average particle size of about 1.7 microns.

In some embodiments, depending on the column dimension chosen and optimization necessary, the flow rate of the mobile phase is set between about 0.1 mL/min to 4 mL/min, with a backpressure regulator setting to maintain or produce CO₂ is supercritical state, e.g., at about 1000-9000 psi. In other embodiments, the backpressure regulator setting is about 1000-3000 psi. In other embodiments, the backpressure regulator setting is about 1885 psi. In certain other embodiments, temperature may be adjusted to optimize separations with a practical working range of 5° C. to 85° C.

As described herein, at least one contributing factor for the superior separations achieved with one or more fatty acids is the ability to use, and the inclusion of, smaller average particle sized columns. It was discovered that the use of smaller average particles sized led to efficient and superior separation of one or more fatty acids from a sample mixture. This effect is based, in part, on the discovery that improved pressure stability is one of the advantageous features that the presently described CO₂-based chromatography systems provide. That is, the backpressure and/or operating pressures of the system are effectively held (or maintained) at constant rate without pressure variations or drops that would otherwise effect separation of the sample. Also, it would be understood that in the event of pressure gradients, the CO₂-based systems described herein effectively hold (or maintain) the backpressure and/or operating pressures at which the pressure is constant for the specified time.

### The Mobile Phase Solvent and Sample Solution

Due to the fact that CO₂ is miscible with solvents having a variety of elution power, various polar and non-polar co-solvents can be added to CO₂ to facilitate desorption of one or more fatty acids. A related advantage of the CO₂-based chromatography system is its compatibility with a wide range of sample solutions.

The sample solution can comprise water, an aqueous solution, or a mixture of water or an aqueous solution, a water-miscible polar organic solvent, non-polar solvents such as alkane based solvents, chlorinated solvents, or a mixture of polar and non-polar miscible solvents. e.g., methanol, ethanol, N,N-dimethylformamide, dimethylsulfoxide, 2-propanol, acetonitrile, hexane, heptanes, methylene chloride, chloroform, and methyl tertiary butyl ether (MTBE). The latter MBTE providing good solubility for the one or more fatty acid separations. In an embodiment, the solution is an acidic, basic or neutral aqueous, i.e., between about 1% and about 99% diluent by volume, solution.

In some embodiments, CO₂ is used as the primary mobile phase solvent. Due to its miscibility, the CO₂ solvent can be combined with one or more modifiers (co-solvents) for more effective desorption or elution of the one or more fatty acids from the chromatographic column. In some embodiments, suitable modifiers that are combined with CO₂ are, e.g., polar water-miscible organic solvents, such as alcohols, e.g., methanol, ethanol or isopropanol, acetonitrile, acetone, and tetrahydrofuran, or mixtures of water and these solvents. The modifiers can also be, e.g., a nonpolar or moderately polar water-immiscible solvent such as pentane, hexane, heptane, xylene, toluene, dichloromethane, diethylether, chloroform, acetone, doxane, THF, MTBE, ethylacetate or DMSO. Mixtures of these solvents are also suitable. In some embodiments, modifiers or modifier mixtures must be determined for each individual case. A suitable modifier can be determined by one of ordinary skill in the art without undue experimentation, as is routinely done in chromatographic methods development.

In an embodiment, the ratio of a modifier to CO₂ (v/v) is between about 0.0001 to 1 to about 1 to 1. In another embodiment, this ratio of a modifier to CO₂ (v/v) is between about 0.001 to 1 to about 1 to 1, or any ratios in between. In another embodiment, the modifier is being added to CO₂ in a gradient during the CO₂-based chromatography system run time and/or during the column elution period. In some embodiments, the mobile phase flow is in gradient, i.e., the flow volume decreases or increases with time. In an exemplary embodiment, a combination of methanol with about 0.3% isopropyl amine is used as a modifier, under gradient conditions of about 2% to 13% (v/v to CO₂) in 2 minutes, with a simultaneous flow gradient of about 3.0 mL/min to 2.5 mL/min. In some embodiments the modifier gradient is from about 1% to 100% during the elution period. In some embodiments, the mobile phase flow gradient is from about 3.0 mL/min to 1.5 mL/min, or any specific rate within this range. In some other embodiments, the mobile phase flow gradient is from 3.0 mL/min to 5.0 mL/min, or any specific rate within this range.

In some embodiments, depending on the nature of the fatty acid or modification of the fatty acid, the method conditions can be further modified to optimize the separation. In an embodiment, organic modifiers including methanol, ethanol, isopropanol or acetonitrile are used alone or in combination with other basic additives (e.g. isopropyl amine, diethyl amine, or ammonium hydroxide). In another embodiment, depending on the polarity of the fatty acid or fatty acid derivative, the modifier concentrations are adjusted from 0% to 40% modifier, in addition to varying the gradient duration (tg).

### Kits and Computer Mediums

Kits for quantifying the one or more fatty acids obtained by the CO₂-based chromatography methods and apparatus described herein are also provided. In one embodiment, a kit may comprise a first known quantity of a first calibrator, a second known quantity of a second calibrator, and optionally comprising one or more fatty acids, wherein the first known quantity and the second known quantity are different, and wherein the first calibrator, the second calibrator, and the one or more fatty acids are each distinguishable in a single sample by mass spectrometry.

The kits described herein may also comprise instructions for: (i) obtaining a mass spectrometer signal comprising a first calibrator signal, a second calibrator signal, and one or more fatty acids from the single sample comprising the first known quantity of the first calibrator, the second known quantity of the second calibrator, and optionally comprising one or more fatty acids; and (ii) quantifying one or more fatty acids in the single sample using the first calibrator signal, the second calibrator signal, and the signal of the one or more fatty acids.

In some embodiments, the first calibrator and the second calibrator are each analogues, derivatives, metabolites, or related compounds of the one or more fatty acids.

Kits may also comprise a third known quantity of a third calibrator and a fourth known quantity of a fourth calibrator, wherein the third known quantity and the fourth known quantity are different, and wherein the first calibrator, the second calibrator, the third calibrator, the fourth calibrator, and the one or more fatty acids are each distinguishable in a single sample by mass spectrometry. These kits may also further comprise instructions for: (i) obtaining a mass spectrometer signal comprising a third calibrator signal, a fourth calibrator signal, and one or more fatty acids from the single sample comprising the third known quantity of the third calibrator, the fourth known quantity of the fourth calibrator, and optionally comprising one or more fatty acids; and (ii) quantifying one or more fatty acids in the single sample using the third calibrator signal, the fourth calibrator signal, and the signal of the one or more fatty acids.

The kits described herein may further comprise additional calibrators, such as, e.g., from 5 to 10 calibrators including both nonzero and blank calibrators. Instructions for obtaining mass spectrometer signals and quantifying one or more fatty acids using these additional calibrators is also contemplated. In one exemplary embodiment, the kit contains 6 nonzero calibrators and a single blank calibrator.

Computer readable mediums for use with the CO₂-based chromatography methods and apparatus are also provided. In an exemplary embodiment, a computer readable medium may comprise computer executable instructions adapted to: separating one or more fatty acids as described herein and obtaining a mass spectrometer signal comprising a first known quantity of a first calibrator, a second known quantity of a second calibrator, and optionally comprising one or more fatty acids, wherein the first known quantity and the second known quantity are different, and wherein the first calibrator, the second calibrator, and the one or more metabolites are each distinguishable in a single sample by mass spectrometry.

The computer readable medium may further comprise executable instructions adapted to quantifying one or more fatty acids in the single sample using the first calibrator signal, the second calibrator signal, and the signal of the one or more fatty acids.

While the subject technology has been particularly described with reference to the various figures and configurations, it should be understood that these are for illustration purposes only and should not be taken as limiting the scope of the subject technology. There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

The subject technology is further illustrated by the following examples which should not be construed as limiting. The contents of all references, patents and published patent applications cited throughout this application, are incorporated herein by reference.

The unique speed and resolution provided by the CO₂-based chromatography system described herein allows for conducting fatty acid assays that are rapid enough to use for routine screening and diagnostic testing. As discussed herein, the present disclosure is based, in part, on the discovery that the CO₂-based chromatography system of the present disclosure provided a rapid separation of multiple closely related fatty acids in less than about 2 minutes. As shown in FIG. 4, even at such a short run time, the peaks associated with the fatty acids were well-resolved. These results are attributable to the CO₂-based chromatography system described herein, and the column chemistry and stationary phase particle sizes used therein. Some of these attributes are discussed below.

### EXAMPLES

### Example 1

### Rapid Analysis of Fatty Acids

A sample of nine closely related fatty acids (ranging from C₈ to C₂₄) were prepared and injected into a CO₂-based chromatography system as described herein. Analysis was performed using high strength silica particles having an average particle size of about 1.7 microns (ACQUITY UPC²® HSS C18 SB Column (3.0×100 mm), Waters Corporation, Milford Mass.) with mass spectrometry detection. Injection volume was 0.5 uL with a gradient run of 1 to 10% over 5 minutes with MeOH w/2 g/L ammonium formate as modifier. Flow and temperature were set to 2.5 mL/min and 60° C., respectively. Make-up flow comprised 0.2 mL/min of 0.1% formic acid. The backpressure of the backpressure regulator was set to approximately 1885 psi.

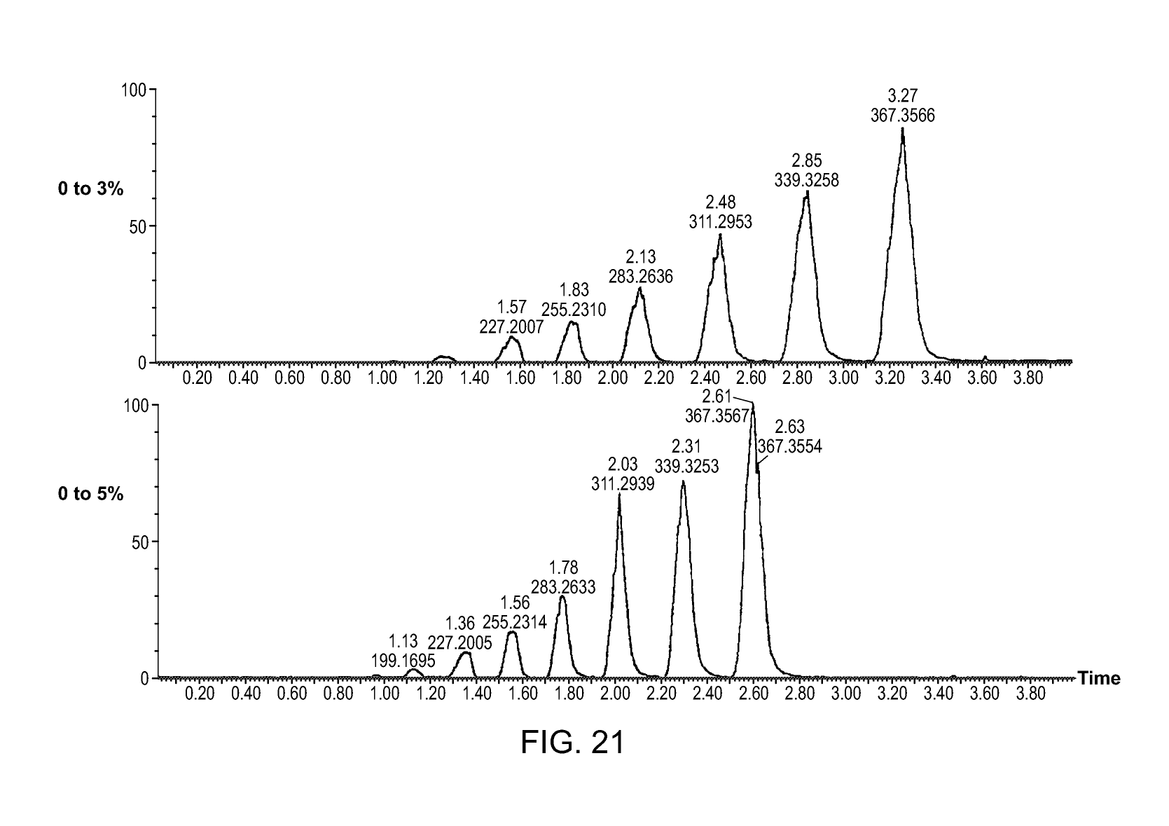

The results shown in FIG. 19 show that baseline resolution of the nine fatty acids was achieved in approximately 2 minutes. It should be noted that the total run time can also be shortened to approximately 1.5 minutes when the gradient slope is maintained. These results demonstrate that the CO₂-based chromatography system described herein provide a rapid and efficient method for separating and detecting fatty acids.

### Example 2

### Pressure Gradient Effects

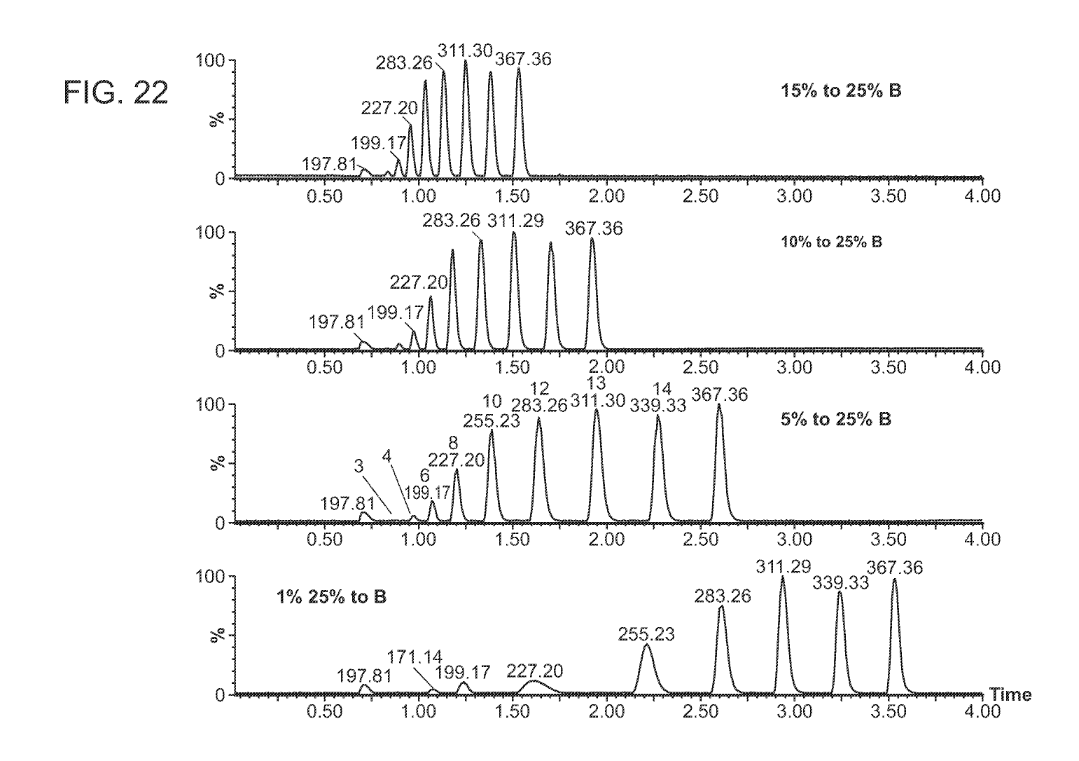

FIG. 20 represents the effect on backpressure on the same mix of fatty acids in Example 1. As shown, the analysis was run under the same method of Example 1, except that the method was performed isocratically at 2% co-solvent and the backpressure of the backpressure regulator was varied from about 1500 to about 3000 psi linearly during the run time. Interestingly, it was found that efficient separation of the fatty acids resulted even at lower pressures. For example, the C₁₀ and C₁₂ fatty acids were not seen at higher pressure. More specifically, it was found that the lowest pressures (about 1500 and about 2000 psi), without going past the cut-off at which the CO₂ is no longer compressed and beyond the boiling point, increased retention time and sensitivity was achieved.

Thus, these results further demonstrate the varying impact of pressure on the separation of fatty acids and, as such, represent the superior results obtained when using the present CO₂-based chromatography systems, which comprise amongst other features, improved pressure stability.

### Example 3

### Varying Solvent Gradient Conditions

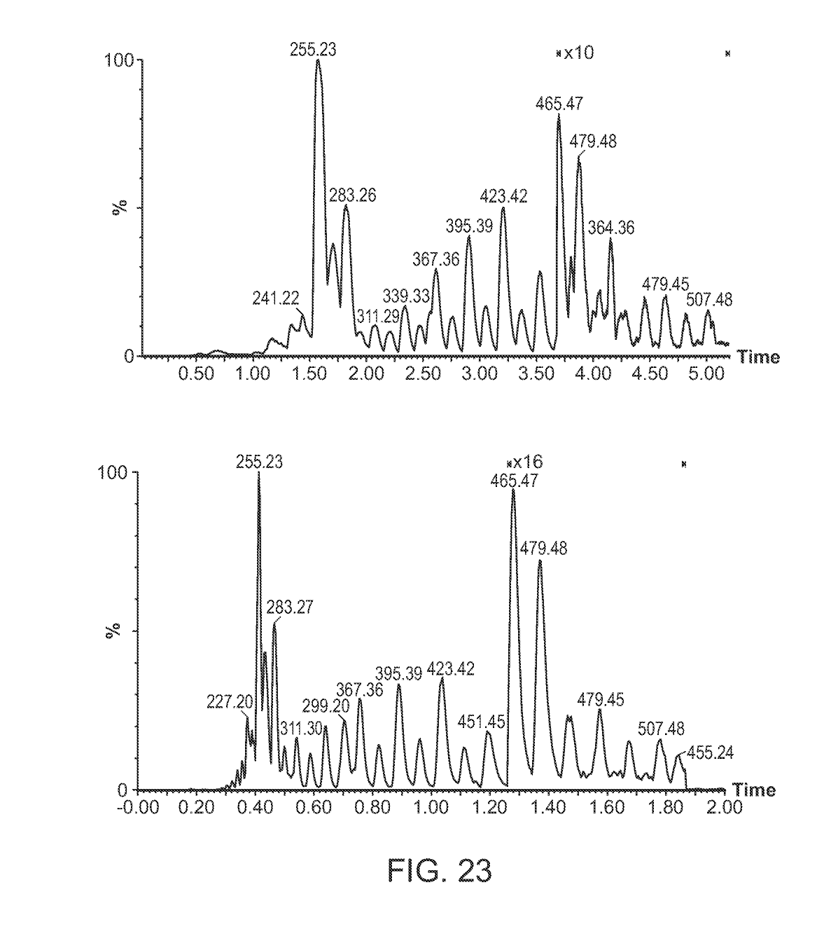

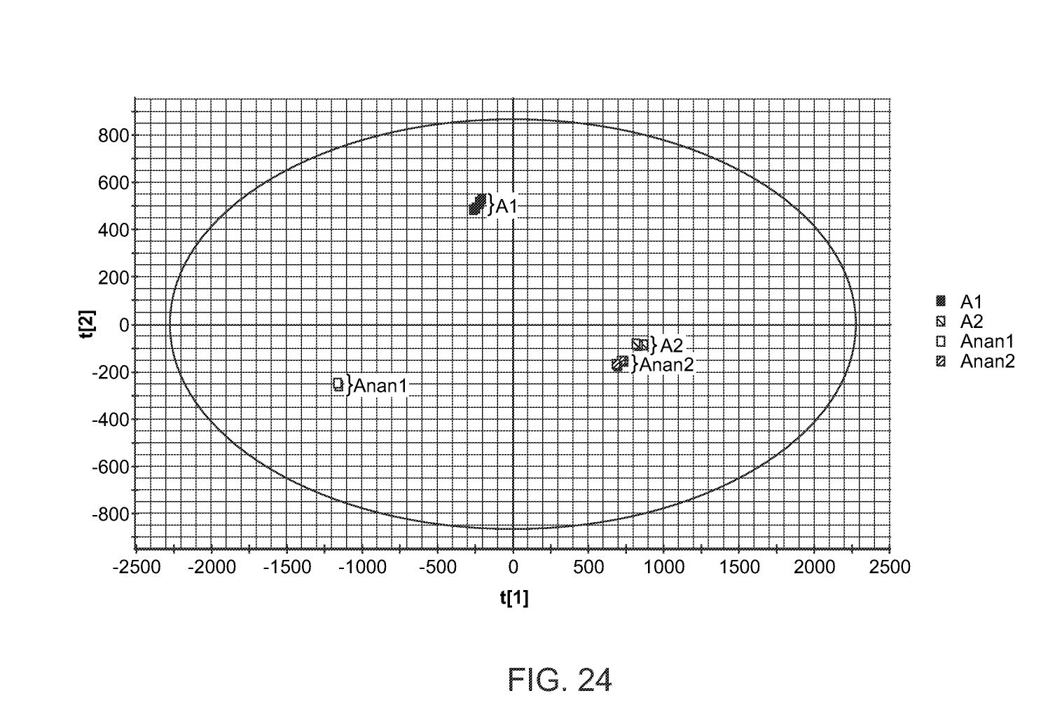

As shown by FIGS. 21 and 22, different solvent gradient effects on the resolution of a panel of fatty acids were investigated. While the goal was to increase the separation between the fatty acids, it was initially found that gradient conditions comprising low concentrations of co-solvent had minimal impact. For example, a gradient of 0 to 3% over 5 min only slightly affected peak shape and resolution when compared to a gradient of 0 to 5% (FIG. 21). Thus, even at low concentrations of co-solvent, effective separation of the panel of fatty acids was achieved. Minimal use of co-solvent(s) generates less waste for disposal and reduces the cost of analysis per sample. Additionally, in cases where e.g., the desired analyte may not be stable under higher concentrations of co-solvent, or where e.g., a particular chromatographic packing material may not be robust enough to handle higher percentages of co-solvent, the instant apparatus and methods are shown to produce effective separations even at low co-solvent concentrations.

In the alternative, the methods and apparatus describe herein may also be effectively employed at higher percentages of co-solvent where e.g., higher percentages of co-solvent would not be expected to have adverse affects on a particular analyte or packing material, or e.g., where disposal of waste and/or reduced cost(s) are inevitable. As shown in FIG. 22, higher percentages of co-solvent produced shorter retention times and narrower peaks without drastically affecting the ability to separate and identify the panel of fatty acids. In addition, under conditions where a reduction of co-eluting lipid species is desired, increasing the range of the amount of co-solvent (e.g., 5% to 25% or 1% to 25%) was shown to effectively increase peak capacity.

The above data shows the versatility of the apparatus and methods described herein in that, in one aspect, effective separations of fatty acids were achieved under various solvent gradient conditions (e.g., with different ranges and amounts of co-solvent). Thus, depending upon the properties of the analyte (such as a fatty acid), or other factors such as the robustness of a particular chromatographic column packing material, solvent gradient condition can be manipulated without drastically affecting the overall separation of the desired product or material. It is postulated that such versatility in achieving effective separations under various gradient conditions with the methods and apparatus disclosed herein is attributed to the combined unique properties (such as, e.g., smaller average particle sized column(s), CO₂-based method(s), mechanically self calibrating needle valve(s), force balance needle valve pressure regulator(s), low volume, pressure assisted, stem and seat vent valve(s), and combined dynamic and static pressure regulators(s)), which allow for enhanced pressure stability on the separation and resolution of the panel of fatty acids.

### Example 4

### Separation of Biopolymers

The profile of free fatty acids (FFA) in algae and algaenan extracts was achieved using the methods and apparatus described herein.

Oil produced from hydrous pyrolysis of algae and algaenan at low and high pyrolysis temperature were provided from Old Dominion University (Norfolk, Va., USA). Algae 1 and algaenan 1 were treated at a pyrolysis temperature of (310° C.) and algae 2 and algaenan 2 were treated at a pyrolysis temperature of (360° C.). Lipids were removed from the algae by Soxhlet extraction with 1:1 (v/v) benzene/methanol solvent mixture for 24 hours. The residue was treated with 2N sodium hydroxide at 60° C. for two hours. The remaining residue was then washed excessively with deionized water, followed by treatment with Dowex 50W-x8 cation exchange resin to exchange any residual sodium. The solid was rinsed with deionized water and the oil samples were diluted 10 times in dichloromethane.

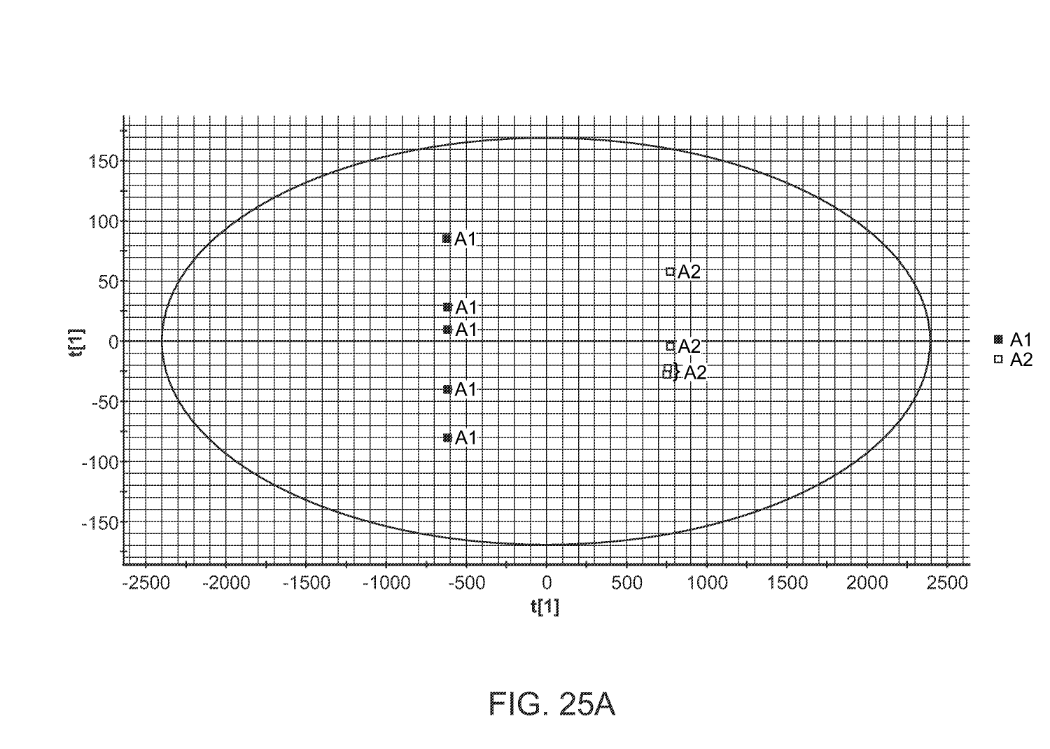

FIG. 23 shows a representative chromatogram from algaenan 1 using high strength silica particles having an average particle size of about 1.8 microns (ACQUITY UPC²® HSS C18 SB Column, Waters Corporation, Milford Mass.) with mass spectrometry detection. Injection volume was 0.5 uL with a gradient run of compressed CO₂ with top 1 to 10% over 10 minutes MeOH with 0.1% formic acid, lower 5% to 20% MeOH with 0.1% formic acid. Flow and temperature were set to 0.6 mL/min and 50° C., respectively. Make-up flow comprised 0.2 mL/min of 0.1% NH₄OH.

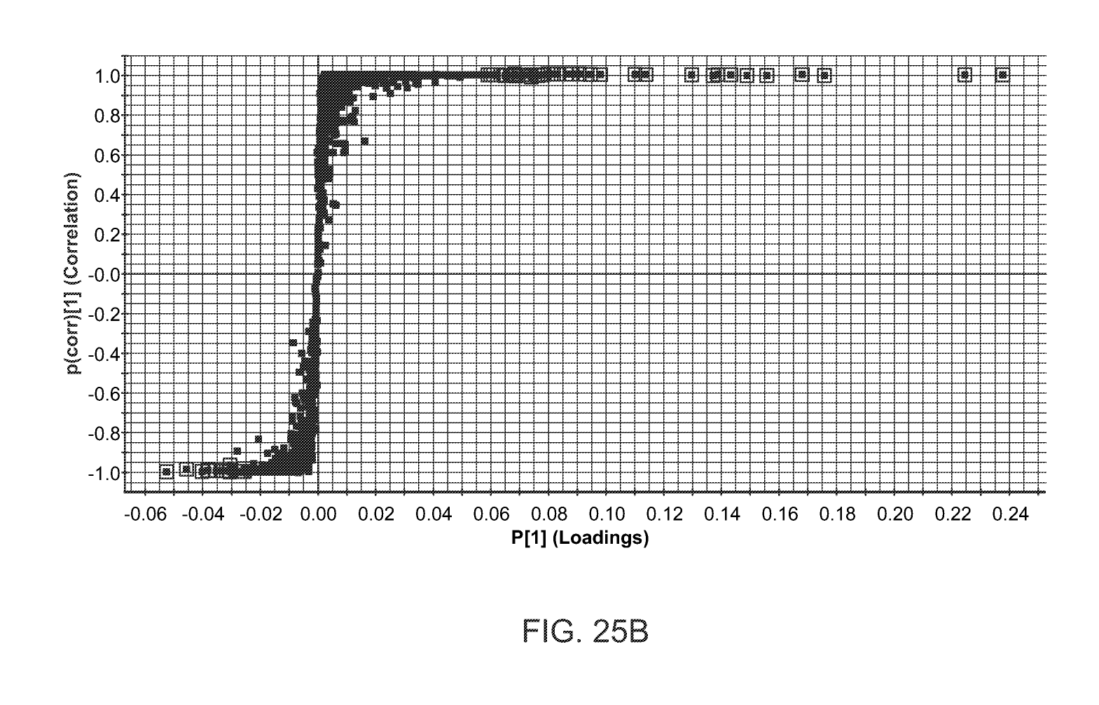

Lipid profiles of the algae and algaenan oil were further investigated using informatics software (TransOmics software available from Nonlinear Dynamics) to determine the pattern and composition of FFA at two different pyrolysis temperatures. Differential analysis of results across different treatments can quickly be performed, thereby facilitating identification and quantitation of potential biomarkers. In FIG. 24, Principal Component Analysis (PCA) was used in the first instance to identify the combination of the FFA species that best describe the maximum variance between algae 1, algae 2, algaenan 1, and algaenan 2 oils. The PCA plot showed excellent technical measurements for the CO₂-based chromatography methods and apparatus described herein. The PCA plot effectively displays the inter-sample relationships in multi-dimensional hyperspace, with more similar samples clustering together and dissimilar samples separated. The clustering in FIG. 24 indicates that algae 1 and algaenan 1 are different, but algae 2 and algaenan 2 have more similarity in their FFA compositions after high pyrolysis temperature treatment. Orthogonal projections latent structure discriminant analysis (OPLS-DA) binary comparison can be performed between the different sample groups (algae 1 vs. algae 2, algaenan 1 vs. algaenan 2, algae 1 vs. algaenan 1, and algae 2 vs. algaenan 2) to find out the features that change between the two groups.

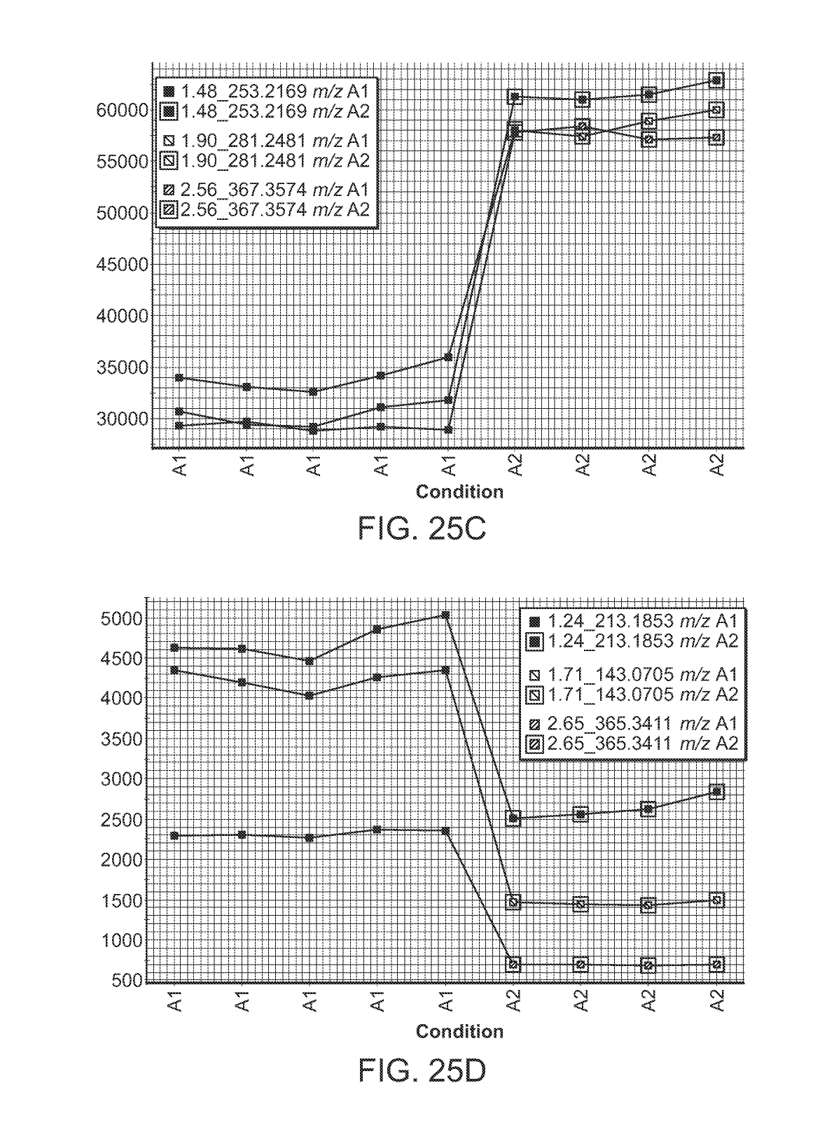

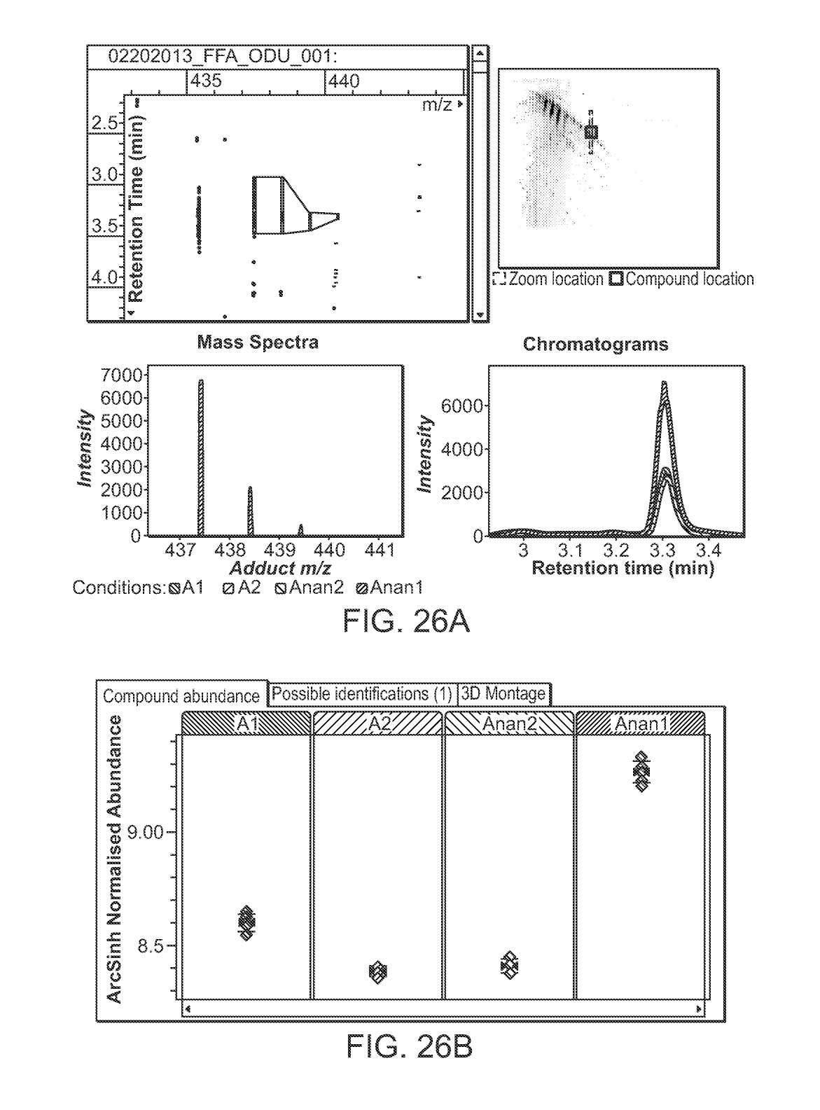

As an example, the OPLS-DA binary comparison between algae 1 vs. algae 2 is shown in FIG. 25a. As shown in the S-plot, the features that contribute most to the variance between the two groups are those farthest from the origin of the plot, highlighted by rectangular points (FIG. 25b). FIGS. 25c and 25d show representative trend plots that change most between algae 1 and algae 2. FIG. 26a shows the ion map, mass spectrum, and chromatogram across all the runs for FFA 29:0. This view allows to review compound measurements such as peak picking and alignment to ensure they are valid across all the runs. FIG. 26b shows the normalized abundance of FFA 29:0 across all the conditions. FFA 29:0 is elevated in algeanan 1 compared to algae 1, algae 2, and algeanan 2; however, there is no significant difference between algae 2 and algeanan 2. Investigation and comparison between algae 1 and algae 2 showed that algae 1 contains elevated levels of short (C9:0 to C13:0) and long (C31:0 to C37:0) chain FFA, whereas algae 2 contains elevated levels of medium (C14:0-C29:0) chain FFA. Similarly, the comparison between algaenan 1 and algaenan 2 showed that algaenan 1 contains elevated levels of long (C28:0 to C37:0) chain FFA, whereas algaenan 2 contains elevated levels of short and medium (C9:0 to C27:0) chain FFA.

The data described above further shows the advantage of the present apparatus and methods for elucidating and separating free fatty acid profiles from biological based samples, e.g., in biopolymers. The added versatility of being able to interface the present apparatus and methods to various software methods, such as TransOmics for Metabolomics and Lipidomics, facilitates an unique workflow approach to performing comparative data analysis.

### Example 5

### Examination of Phospholipids and Sphingolipids in Mouse Heart Extract

Examination of mouse heart extract was performed for rapid inter-class targeted screening of lipids with different polarity. Chloroform/methanol (2:1) was added to a final volume 20 fold the volume of the original tissue sample (e.g. 0.05 g in 1.0 mL of solvent mixture). The heart tissue was then dispersed using a homogenizer and the mixture was vortexed or agitated for 30 min at room temperature in a shaker. The homogenate was centrifuged at 9000×g for 10 min, the supernatant was recovered and transferred to a new glass tube via glass pipette. The supernatant was washed with 0.2 volumes (e.g. 0.2 mL for 1.0 mL) of water to remove salts and any waters soluble metabolites. The sample was vortexed for 30 s and then centrifuged at 1000×g for 5 min to separate the two phases. The upper aqueous phase was then discarded. Exemplary experimental methods for this sample preparation can be found in Folch et al., J. Biol. Chem. 1957, 226, 497-509.

Analysis was performed using ethylene bridged hybrid particles having an average particle size of about 1.7 microns (ACQUITY UPC² BEH C18 SB column, Waters Corporation, Milford Mass.) with mass spectrometry detection. Injection volume was 1.0 uL with a gradient run of compressed CO₂ with 15 to 50% over 3 minutes with 1:1 MeOH/CH₃CN w/1 g/L ammonium formate as modifier, held at 1:1 MeOH/CH₃CN for 2 minutes. Flow and temperature were set to 1.85 mL/min and 60° C., respectively. The backpressure of the backpressure regulator was set to approximately 1500 psi.

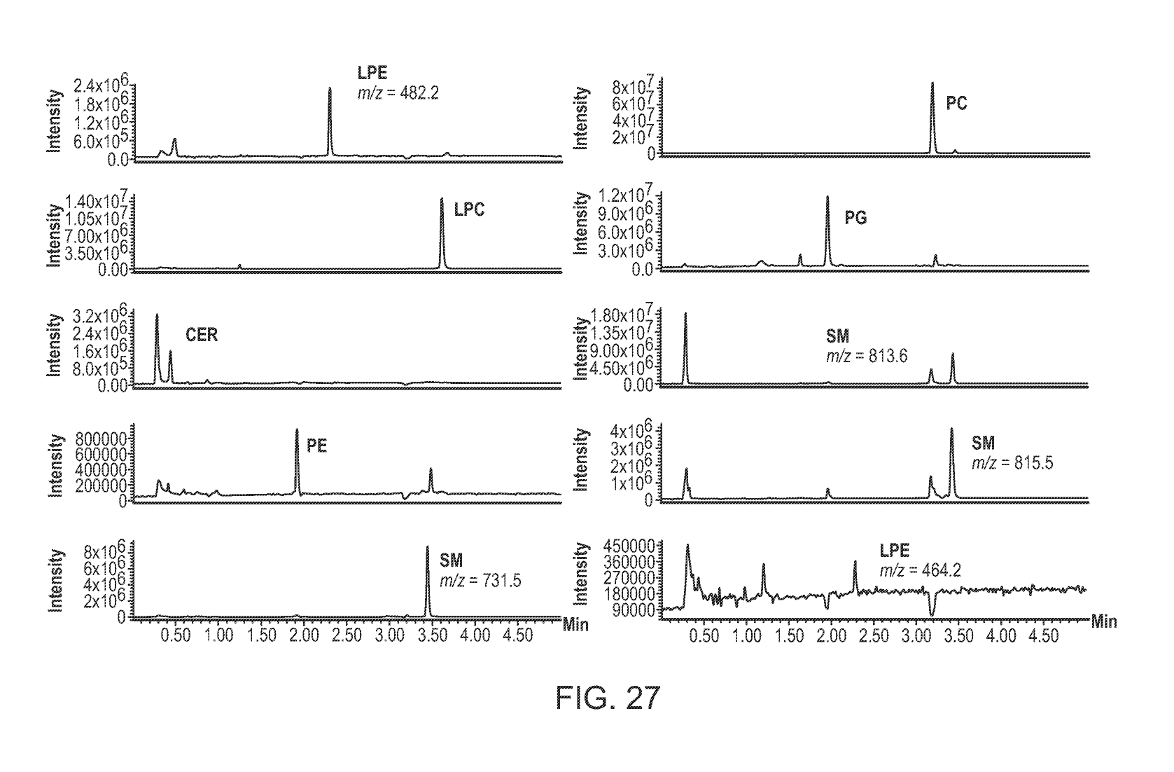

As shown by FIG. 27, many of the phospholipids and sphingolipids were easily identified in the mouse heart extract, where LPE=lyso-phosphatidylethanolamine, LPC=lyso-phosphatidylcholine, CER=ceramides, PE=phosphatidylethanolamine, PC=phosphatidylcholine, PG=phosphatidylglycerol, and SM=sphingomyelin.

### Example 6

### Analysis of Blood Samples

Blood samples were obtained as dried spots on Whatman filter paper. The Free Fatty Acids (FFA) were either extracted directly from the filter paper (FFA), or as Fatty Acid Methyl Esters (FAME). The extraction methods are specified below.

FAME:

1 ml of 0.5M HCl in methanol+100 μl of Internal standard was added to the blood spots, mixed and incubated at 70° C. for 1 hour. After cooling 1 ml of de-ionized water and 1 ml of saturated potassium chloride was added and thoroughly mixed. 2 ml of hexane was added, mixed, and centrifuged for 5 minutes. The sample was frozen in liquid nitrogen and the hexane layer was transferred into a new vial and dried under nitrogen at 40° C. for 20 minutes. The sample was re-dissolved in 50 μl of hexane. 1 μl was injected for analysis.

FFA:

400 μl of saline solution was added to blood spots and left at room temperature for 20 minutes with intermittent shaking. 1 ml of hexane was added and after vigorous mixing the vial was centrifuged for 5 minutes. The hexane layer was aspirated into a new vial and dried under nitrogen. The sample was redissolved in 100 μl of hexane. 2 μl was injected for analysis.

Analysis was performed using high strength silica particles having an average particle size of about 1.7 microns (ACQUITY UPC²® HSS C18 SB Column (3.0×100 mm), Waters Corporation, Milford Mass.) with mass spectrometry detection. The eluent composition was as eluent A, compressed CO₂, and as eluent B, 0.2% (w/v) ammonium formate in methanol. Gradients were as follows.

FAME-Analysis:

The gradient used was 0-0.1 min 0% B, 0.1-0.7 min 0%-0.4% B, 0.7-1.0 min 0.4%-0.7% B, 1.0-1.3 min 0.7%-8% B, 1.3-1.4 min 8%-30% B, 1.4-2.4 min 30% B, 2.4-2.5 min 30%-0% B. 2.5-3.5 min 0% B. The time between injections was 3.5 minutes.

FFA-Analysis:

The gradient used was 0-0.1 min 2.5% B, 0.1-2.0 min 2.5%-4.0% B, 2.0-2.2 min 4.0%-30% B, 2.2-3.2 min 30% B, 3.2-3.3 min 30%-2.5% B. 3.3-4.5 min 2.5% B. The time between injections was 4.5 minutes.

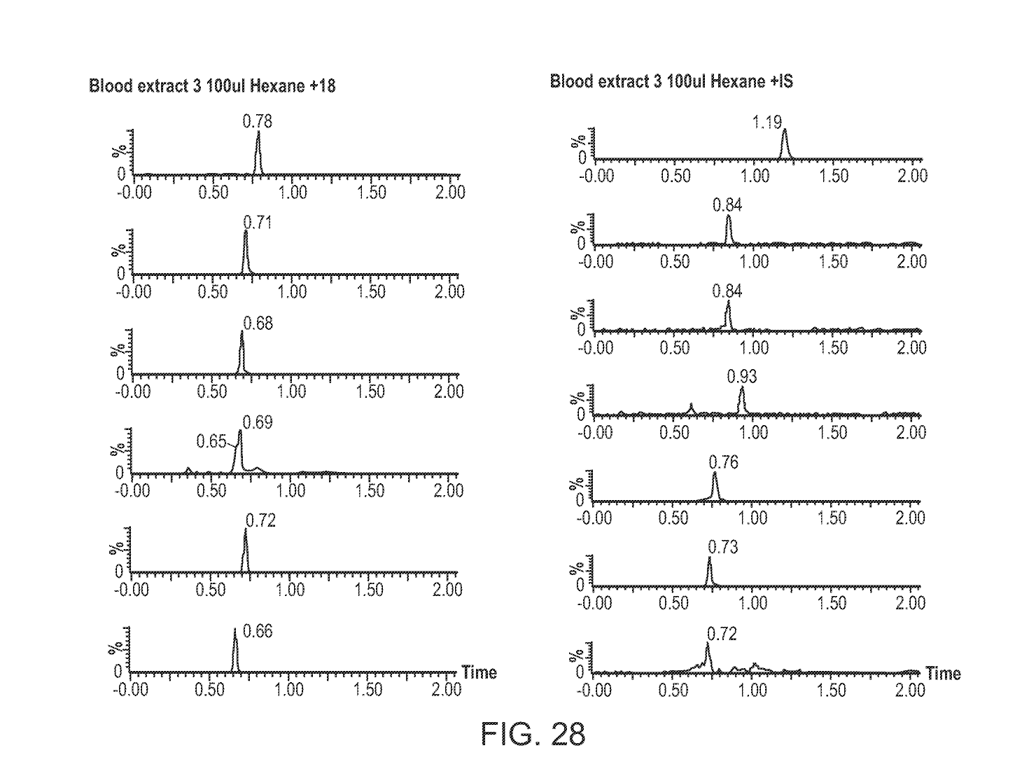

FIG. 28 shows the chromatograms from the extracted blood sample using FAME sample preparation. The above data shows that the present disclosure further provides methods to analyze fatty acid methyl esters in dried blood spot samples.

### Example 7

### Analysis of Oils

Triacylglycerols (TAGs) in peanut, sunflower seed, and soybean oil were separated using high strength silica particles having an average particle size of about 1.7 microns (ACQUITY UPC² HSS C18 SB column (3.0×150 mm), Waters Corporation, Milford Mass.) with mass spectrometry detection. Injection volume was 1.0 uL with a gradient run of 3% CH₃CN for 2 minutes then linear gradient to 70% CH₃CN in 15 min, then hold at 70% CH₃CN for 5 minutes. Flow and temperature were set to 1.0 mL/min and 20° C., respectively. The backpressure of the backpressure regulator was set to approximately 1500 psi.

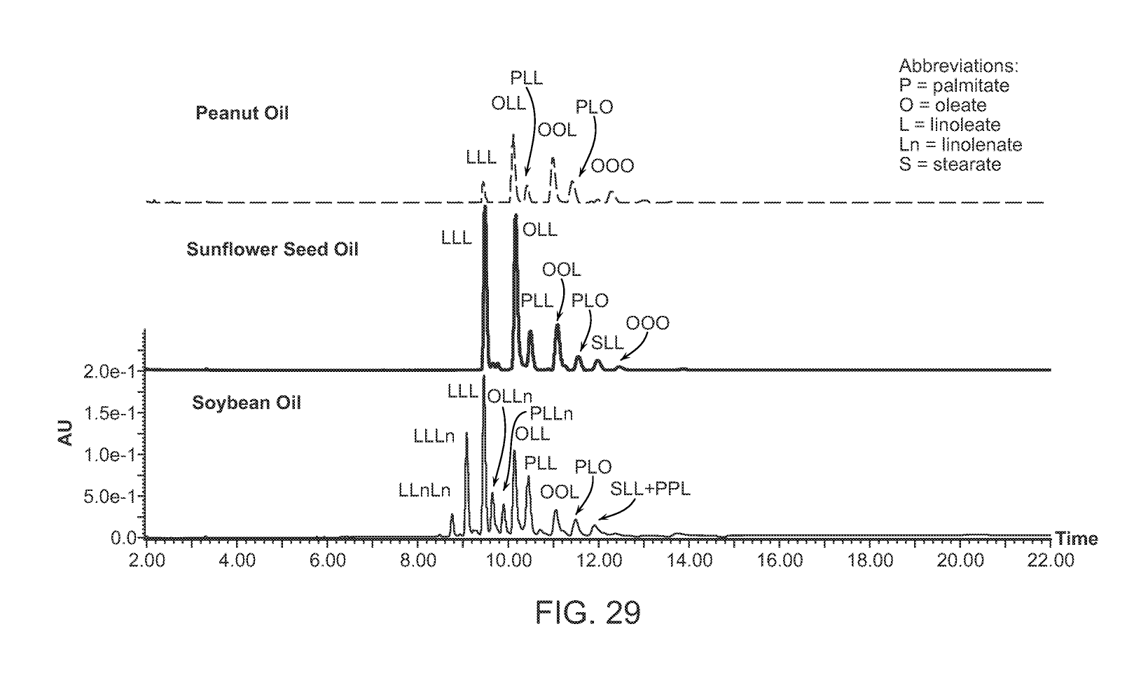

FIG. 29 shows the UV chromatograms (210 nm) of TAGs in peanut, sunflower seed, and soybean oils. All TAGs eluted in 15 minutes and showed baseline separation for all the major TAGs. This approach is significantly faster than conventional non CO₂-based methods, which typically take 30 to 80 minutes.

### Example 8

### Analysis of Impurities in Model Biodiesel

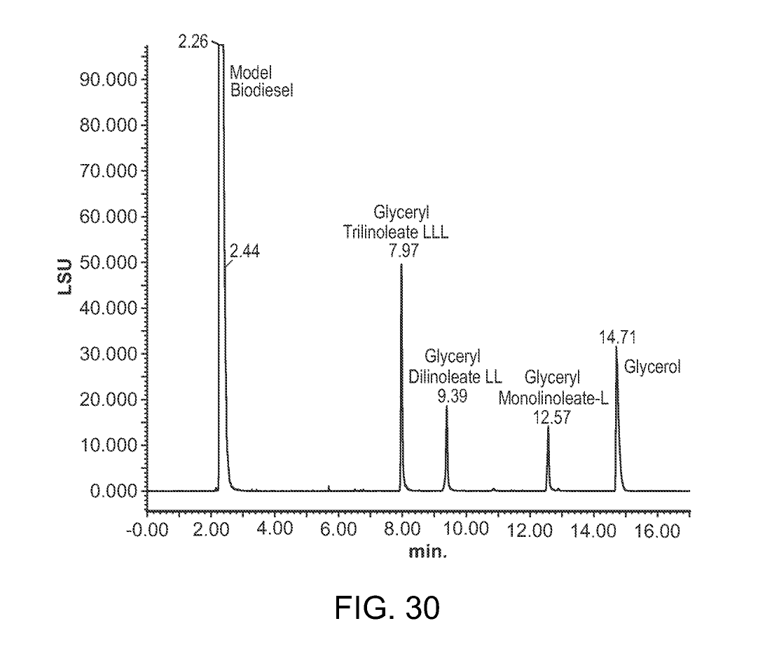

Pure fatty acid ethyl esters were purchased from Sigma-Aldrich (St. Louis, Mo.) and mixed to form a model biodiesel. Mono-acylglycerol, di-acylglycerol, and tri-acylglycerol plus glycerol and soybean oil were also obtained from Sigma-Aldrich. Standards were prepared in 1:1 DCM/MeOH and model biodiesel was prepared as a 5% (w/w) solution. Glycerol, soybean oil acylglycerols, and model biodiesel components were separated using high strength silica particles having an average particle size of about 1.8 microns (ACQUITY UPC² HSS C18 SB column (3.0×150 mm), Waters Corporation, Milford Mass.) with mass spectrometry detection. Injection volume was 2-8 uL with a gradient run of 98:2 (compressed CO₂: CH₃CN/methanol (90:10)) to 80:20 (compressed CO₂: CH₃CN/methanol (90:10)) in 18 min. Flow and temperature were set to 1.0-2.0 mL/min and 25° C., respectively. The backpressure of the backpressure regulator was set to approximately 1500 psi. FIG. 30 shows effective baseline separation of all the components.

The specification should be understood as disclosing and encompassing all possible permutations and combinations of the described aspects, embodiments, and examples unless the context indicates otherwise. One of ordinary skill in the art will appreciate that the invention can be practiced by other than the summarized and described aspect, embodiments, and examples, which are presented for purposes of illustration, and that the invention is limited only by the following claims.

1. A method of separating one or more fatty acids, comprising:
placing a sample in a CO₂-based chromatography system comprising a chromatography column having an average particle size of 2 microns or less; and
eluting the sample by a gradient of organic solvent and a mobile phase fluid comprising CO₂ to substantially resolve the one or more fatty acids, wherein:
the CO₂-based chromatography system comprises an operating system pressure of about 1000 to about 9000 psi and a backpressure of about 1000 to about 9000 psi; and one or more pumps for delivering a flow of the mobile phase fluid comprising CO₂; provided that
the operating system pressure and back pressure are each individually essentially void from fluctuations throughout the separation of the one or more fatty acids.

2. The method of claim 1, wherein at least a portion of the CO₂ is in or near supercritical state.

3. The method of claim 1, wherein the chromatography column comprises particles having an average particle size of about 1.7 or about 1.8 microns.

4. The method of claim 1, wherein the operating pressure and back pressure are each individually essentially void of pressure differentials that suppress the resolution of the one or more fatty acids.

5. The method of claim 1, wherein the backpressure is about 1000 to 3000 psi.

6. The method of claim 1, wherein the sample is a biological sample.

7. The method of claim 1, wherein the sample is a commercial sample.

8. The method of claim 1, wherein the chromatography column comprises an internal diameter of about 3.0 and a length of about 100 mm.

9. The method of claim 1, wherein the retention times of the one or more fatty acids range from about 0.5 to about 2 minutes.

10. The method of claim 1, wherein the total elution time is less than about 3 minutes.

11. The method of claim 1, wherein the fatty acids comprise aliphatic tails comprising from about 6 to about 26 carbon atoms.

12. The method of claim 1, wherein the CO₂-based chromatography system is coupled to a Mass Spectrometer.

13. A method of separating one or more fatty acids, comprising:
placing a sample in a CO₂-based chromatography system comprising a chromatography column having an average particle size of about 1.7 or about 1.8 microns; and
eluting the sample by a gradient of organic solvent and a mobile phase fluid comprising CO₂ to substantially resolve the one or more fatty acids, wherein at least a portion of the CO₂ is in or near supercritical state.

14. The method of claim 13, wherein the CO₂-based chromatography system comprises an operating system pressure and a back pressure, each of which individually being essentially void from fluctuations throughout the separation of the one or more fatty acids.

15. The method of claim 13, wherein the CO₂-based chromatography system comprises an operating system pressure and a back pressure, each of which individually being essentially void of pressure differentials that suppress the resolution of the one or more fatty acids.

16. The method of claim 13, wherein the chromatography column comprises high strength silica particles or ethylene bridged hybrid particles optionally comprising one or more diol ligands.

17. The method of claim 13, wherein the chromatography column comprises an internal diameter of about 3.0, and a length of about 100 mm.

18. The method of claim 13, wherein the retention times range from about 0.5 to about 2 minutes.

19. The method of claim 13, wherein the total elution time is less than about 3 minutes.

20. The method of claim 13, wherein the fatty acids comprise aliphatic tails comprising from about 6 to about 26 carbon atoms.

21. The method of claim 13, wherein the sample is a biological sample or commercial sample.

22. The method of claim 13, wherein the CO₂-based chromatography system is coupled to a Mass Spectrometer.

23. A computer readable medium comprising computer executable instructions adapted to:
separating one or more fatty acids obtained by the method of claim 1; and
obtaining a mass spectrometer signal comprising a first known quantity of a first calibrator, a second known quantity of a second calibrator, and optionally comprising one or more fatty acids, wherein the first known quantity and the second known quantity are different, and wherein the first calibrator, the second calibrator, and the one or more metabolites are each distinguishable in a single sample by mass spectrometry.

24. The computer readable medium of claim 23, further comprising executable instructions adapted to quantifying one or more fatty acids in the single sample using the first calibrator signal, the second calibrator signal, and the signal of the one or more fatty acids.
